(19) United States
(12) Patent Application Publication
(10) Pub. No.: US 2022/0031990 A1
(43) Pub. Date: Feb. 3, 2022

(54) PATIENT INTERFACE SYSTEM AND COMPONENTS THEREFOR

(71) Applicant: ResMed Asia Pte Ltd, Singapore (SG)

(72) Inventor: Sok Li GOH, Singapore (SG)

(21) Appl. No.: 17/387,226

(22) Filed: Jul. 28, 2021

(30) Foreign Application Priority Data
Jul. 29, 2020 (SG) 10202007224P

(51) Int. Cl. A61M 16/06

(52) U.S. Cl. CPC A61M 16/0683; A61M 2207/00; A61M 2205/0272; A61M 2016/0661

(57) ABSTRACT

A patient interface system which includes a magnetic fastener arrangement to connect a positioning and stabilising structure to a patient interface, a positioning and stabilising structure having at least one strap and a magnetic fattener component provided to the strap. The magnetic fastener component is provided between a distal end of the strap and an anterior portion of the positioning and stabilising structure, and can be formed to the strap.

Methods of manufacturing a positioning and stabilising structure which has at least one strap, where the methods attach or provide a magnetic fastener component to the strap. In forms, the magnetic fastener component is formed to the strap e.g. when forming the strap.

### BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, and their use.

### 1.2 Description of the Related Art

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO₂ to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube. In some forms, the comfort and effectiveness of these therapies may be improved.

### 1.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO₂ from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched gas at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 1.2.2.3 Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

Another form of therapy system is a mandibular repositioning device.

### 1.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 1.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT′ FX nasal pillows).

### 1.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 1.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

Air pressure generators are known in a range of applications, e.g. industrial-scale ventilation systems. However, air pressure generators for medical applications have particular requirements not fulfilled by more generalised air pressure generators, such as the reliability, size and weight requirements of medical devices. In addition, even devices designed for medical treatment may suffer from shortcomings, pertaining to one or more of: comfort, noise, ease of use, efficacy, size, weight, manufacturability, cost, and reliability.

An example of the special requirements of certain RPT devices is acoustic noise.

Table of noise output levels of prior RPT devices (one specimen only, measured using test method specified in ISO 3744 in CPAP mode at 10 cmH₂O).

| | A-weighted sound | Year |
|---|---|---|
| RPT Device name | pressure level dB(A) | (approx.) |
| C-Series Tango ™ | 31.9 | 2007 |
| C-Series Tango ™ with Humidifier | 33.1 | 2007 |
| S8 Escape ™ II | 30.5 | 2005 |
| S8 Escape ™ II with H4i ™ Humidifier | 31.1 | 2005 |
| S9 AutoSet ™ | 26.5 | 2010 |
| S9 AutoSet ™ with H5i Humidifier | 28.6 | 2010 |

One known RPT device used for treating sleep disordered breathing is the S9 Sleep Therapy System, manufactured by ResMed Limited. Another example of an RPT device is a ventilator. Ventilators such as the ResMed Stellar™ Series of Adult and Paediatric Ventilators may provide support for invasive and non-invasive non-dependent ventilation for a range of patients for treating a number of conditions such as but not limited to NMD, OHS and COPD.

The ResMed Elisée™ 150 ventilator and ResMed VS III™ ventilator may provide support for invasive and non-invasive dependent ventilation suitable for adult or paediatric patients for treating a number of conditions. These ventilators provide volumetric and barometric ventilation modes with a single or double limb circuit. RPT devices typically comprise a pressure generator, such as a motor-driven blower or a compressed gas reservoir, and are configured to supply a flow of air to the airway of a patient. In some cases, the flow of air may be supplied to the airway of the patient at positive pressure. The outlet of the RPT device is connected via an air circuit to a patient interface such as those described above.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 1.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 1.2.3.4 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology relates to a positioning and stabilising structure for a patient interface system, comprising:
the fastener arrangement according to any of the forms described herein, and
a headgear assembly comprising one or more headgear straps.

One form of the present technology relates to a patient interface system, comprising:
the position and stabilising structure according to any of the forms described herein; and
a patient interface configured to deliver a supply of pressurised breathable gas to one or more of a patient's airways.

One form of the present technology relates to a method of manufacturing a positioning and stabilising structure for a patient interface system, the method comprising the following steps:
a) providing a first strap layer;
b) providing a second strap layer relative to the first strap layer and the second strap layer;
c) positioning a first fastener half;
   wherein, at least one of step b) or step c) ensure that the first fastener half is located between the first strap layer and the second strap layer; and
d) forming the first strap layer and the second strap layer together to thereby manufacture at least a portion of the positioning and stabilising structure.

In preferred forms, the step of positioning the first fastener half occurs before the step of providing the second strap layer.

In preferred forms, the step of forming the first strap layer and the second strap layer together involves at least one of ultrasonic torsional welding, applying adhesive to one or more of the layers, heat bonding and/or glue potting.

One form the present technology relates to a method of manufacturing a positioning and stabilising structure for a patient interface system, the method comprising the following steps:
a) providing a first strap layer; and
b) attaching a magnetic fastener half directly to the first strap layer.

In preferred forms, the method further comprises the step of positioning a second strap layer, wherein the magnetic fastener half is positioned between the first strap layer and the second strap layer.

In preferred forms, the method further comprises the step of attaching the first strap layer and the second strap layer to each other.

In preferred forms, the method further comprises the step of attaching the first strap layer and the second strap layer to each other, wherein this step involves at least one of heat bonding, torsional ultrasonic welding and/or glue potting.

In preferred forms, the method further comprises the step of selecting the magnetic fastener half.

In preferred forms, the method further comprises the step of creating a guide portion.

In preferred forms, the step of creating the guide portion involves creating a recess in the guide portion which is configured to receive the fastener half.

In preferred forms, the step of creating the guide portion includes radio frequency welding, cutting, pressing or other deformation of the first strap layer.

In preferred forms, the method further comprises forming the first strap layer which comprises a multi-layer structure.

In preferred forms, the method further comprises the multi-layer structure comprises one or more layers selected from the list of a layer of foam, a layer of textile material, and layer of relatively stiffer material e.g. a plastics material which provides a rigidiser.

In preferred forms, the method further comprises the step of attaching two or more layers of material together to create the first strap layer.

In preferred forms, the step of attaching the two or more layers of material together involves laminating the two or more layers of material together.

One form of the present technology relates to a method of manufacturing a patient interface for a treatment system, the method comprising the steps of:
a) providing a first layer of material;
b) providing a second layer of material;
c) positioning a fastener half with respect to the first layer of material;
d) attaching the first layer of material and the second layer of material to each other.

One form of the present technology relates to a method of manufacturing a first fastener half of a positioning and stabilising structure for a patient interface system, the method comprising the following steps:
providing a layer of foam material;
forming a recess in a surface of the layer of foam material;
positioning a spherical-shaped magnet or a cylindrical-shaped magnet in an upright position in the recess, wherein the magnet forms an insertion portion configured to in use magnetically and rotationally engage with a corresponding receiving portion of a second fastener half provided to a patient interface of the patient interface system or another portion of the positioning and stabilising structure; and
attaching a layer of textile material to the layer of foam material such that the cylindrical-shaped magnet is positioned between the layers of material.

One form of the present technology relates to a method of manufacturing a first fastener half of a positioning and stabilising structure for a patient interface system, the method comprising the following steps:
providing a layer of foam material;
forming a recess in a surface of the layer of foam material;
positioning a steel ball in the recess, wherein the steel ball forms an insertion portion configured to in use magnetically and rotationally engage with a corresponding receiving portion of a second fastener half provided to a patient interface of the patient interface system or another portion of the positioning and stabilising structure; and
attaching a layer of textile material to the layer of foam material such that the steel ball is positioned between the layers of material.

One form of the present technology relates to a method of manufacturing a first fastener half of a positioning and stabilising structure for a patient interface system, the method comprising the following steps:
providing a layer of foam and/or textile material;
providing a male component to the layer of foam, fabric and/or textile material;
providing a magnet to the male component, wherein the male component is configured to magnetically and rotationally engage with a complementary female component of a second fastener half, wherein the female component is provided to a patient interface of the patient interface system or another portion of the positioning and stabilising structure;

One form of the present technology relates to a method of manufacturing a portion of a positioning and stabilising structure for a patient interface system, the method comprising the following steps:
providing a layer of foam and/or textile material; and
providing a hollow magnet to the layer of foam and/or textile material, wherein the hollow magnet forms part of a receiving portion configured to in use magnetically and rotationally engage with a corresponding insertion portion of a first fastener half provided to a patient interface of the patient interface system or another portion of the positioning and stabilising structure.

One form of the present technology relates to a method of manufacturing a portion of a positioning and stabilising structure for a patient interface system, the method comprising the following steps:
providing a layer of foam and/or textile material;
providing an eyelet or washer to the layer of foam and/or textile material, wherein the eyelet or washer forms part of a receiving portion configured to in use magnetically and rotationally engage with a corresponding insertion portion of a first fastener half provided to a patient interface of the patient interface system or another portion of the positioning and stabilising structure.

One form of the present technology relates to a method of manufacturing a portion of a positioning and stabilising structure for a patient interface system, the method comprising the following steps:
providing a layer of foam and/or textile material;
providing a female component to the layer of foam and/or textile material, wherein the female component forms a receiving portion configured to in use magnetically and rotationally engage with a corresponding male component of a first fastener half provided to a patient interface of the patient interface system or another portion of the positioning and stabilising structure.

One form of the present technology relates to a method of manufacturing a fastener arrangement of a positioning and stabilising structure for a patient interface system, the method comprising:
any one the steps of the method of manufacturing the first fastener half according to any of the forms described herein; and

any one the steps of the method of manufacturing the second fastener half according to any of the forms described herein.

One form of the present technology relates to a fastener arrangement to attach a positioning and stabilising structure to a patient interface, wherein the fastener arrangement comprises a first fastener half provided to the positioning and stabilising structure and a second fastener half provided to the patient interface, wherein the first fastener half is permanently attached, or formed to, the positioning and stabilising structure, and further wherein the first fastener half and the second fastener half each comprises a magnetic fastener component.

One form of the present technology relates to a treatment system, comprising:
a patient interface to deliver a supply of pressurised breathable gas to one or more of a patient's airways, and
a positioning and stabilising structure;
wherein the positioning and stabilising structure comprises at least one strap and a strap fastener half, wherein the strap fastener half comprises a magnetic fastener component which is formed to the at least one strap, and
the patient interface comprises an interface fastener half which includes a magnetic fastener component, and further wherein
in use the magnetic fastener components together releasably attach the at least one strap to the patient interface.

In preferred forms, the positioning and stabilising structure comprises a first strap fastener half and a second strap fastener half which in use engage with a first patient interface fastener half and a second patient interface fastener half respectively. The first strap fastener half and the second strap fastener half are each a strap fastener half as described herein and the first patient interface fastener half and the second patient interface fastener half are each a patient interface fastener half as described herein. The first strap fastener half and the first patient interface fastener half provide a first magnetic fastener pair, and the second strap fastener half and the second patient interface fastener half provide a second magnetic fastener pair.

In preferred forms, the first magnetic fastener pair are provided on a first lateral side of the patient interface and the second magnetic fastener pair are provided on a second lateral side of the patient interface. Alternatively, the first and second magnetic fastener pairs may be provided on the same lateral side of the patient interface as each other.

One form of the present technology comprises a positioning and stabilising structure for a patient interface system, comprising
a rear strap assembly,
at least one strap which in use extends away from the rear strap assembly and along a side of the patient's face, wherein the at least one strap has a distal end, and
a first strap fastener that comprises magnetic fastener component provided to the at least one strap and that is positioned between the distal end and the rear strap assembly.

One form of the present technology comprises a positioning and stabilising structure for a patient interface system, comprising at least one strap and a strap fastener half, wherein the strap fastener half comprises a magnetic fastener component which is formed to the at least one strap.

One form of the present technology comprises a positioning and stabilising structure for a patient interface system, comprising at least one strap and a strap fastener half which is permanently attached to the positioning and stabilising structure, wherein the strap fastener half comprises a magnetic fastener component, and further wherein the strap fastener half is configured to in use engage with a corresponding fastener half on a patient interface to attach the positioning and stabilising structure to the patient interface.

In preferred forms, the positioning and stabilising structure comprises a first strap and a second strap, and wherein the first strap comprises a first strap fastener half and the second strap comprises a second strap fastener half, and further wherein the first strap fastener half and the second strap fastener half each comprise a magnetic fastener component.

In preferred forms, at least one of the first strap fastener half and the second strap fastener half comprises an insertion portion configured to in use be inserted into a corresponding receiving portion of a patient interface fastener half.

In preferred forms, one of the strap fastener half and the patient interface fastener half comprises a receiving portion, and the other of the strap fastener half and the patient interface fastener half comprises an insertion portion, and wherein in use the insertion portion is inserted into the receiving portion to assist with attaching the positioning and stabilising structure to the patient interface.

In preferred forms, the strap fastener half provides the insertion portion and the patient interface fastener half provides the receiving portion.

In preferred forms, the strap fastener half and the patient interface fastener half have complementary shapes to each other. The complementary shapes allow the insertion portion to be inserted into the receiving portion.

In addition, or alternatively, the receiving portion may be dimensioned to allow the insertion portion to be inserted therein. For instance, the receiving portion may have a width or diameter that is sufficient to accommodate the receiving portion (including any layers of material which may cover the insertion portion).

In preferred forms, the strap fastener half and the patient interface fastener half are able to rotate relative to each other. This may be facilitated by the receiving portion and the insertion portion having complementary shapes to each other e.g. the receiving portion is a cylindrical shaped cavity while the insertion portion has a curved surface provided for instance by a cylindrical or spherical shaped component. Alternatively, the strap fastener half and the patient interface fastener half may be substantially flat.

In preferred forms, the strap fastener half may be attached to a first layer of material. The first layer of material may be a textile material forming part of a strap of the positioning and stabilising structure.

In embodiments, the layer of material may be attached to at least one additional layer of material. The layer of material and the additional layer of material may be laminated, welded, bonded, glued or otherwise attached to each other.

The first layer of material has a patient contacting surface, at least a portion of which lies against a surface of a patient's face and/or head. The additional layer of material provides an outer surface for the relevant part of the positioning and stabilising system which is distal to the patient's skin.

Alternatively, the strap fastener half may be attached to the patient contacting surface of the first layer of material.

In preferred forms, the positioning and stabilising structure comprises at least a first strap and a second strap.

In other forms, the positioning and stabilising structure may also comprise a third strap and a fourth strap. The third strap and fourth strap are configured to in use attach the positioning and stabilising structure to a/the patient interface.

In these embodiments, the third strap and the fourth strap may provide either a pair of upper straps or a pair of lower straps. This facilitates the positioning and stabilising structure having a four-point attachment to the patient interface.

In preferred forms, the first strap fastener half is provided to a first lower strap of the positioning and stabilising structure and the second strap fastener half is provided to a second lower strap of the positioning and stabilising system. In this form, the first patient interface fastener and second the insertion portion and the patient interface fastener half fastener half are positioned on the patient interface in a corresponding location to facilitate engagement with the respective first strap fastener half and the second strap fastener half.

In preferred forms, the first fastener half is partially or completely covered by the first layer of material e.g. the first strap fastener half is on the distal side of the first layer of material from the patient contacting surface.

In preferred forms, the first strap fastener half and the second strap fastener half may provide a self-aligning function. For instance, a magnetic field generated by a/the magnetic component/s of the first strap fastener half and the second strap fastener half, may assist to guide the insertion portion into alignment with the receiving portion. This arrangement may simplify attaching the positioning and stabilising system to the patient interface.

In one form the present technology relates to a method of manufacturing a positioning and stabilising structure for a patient interface system, comprising the following steps in any order:
selecting a first layer of material;
positioning a magnetic fastener component and the first layer of material relative to each other;
attaching the magnetic fastener component to the first layer of material.

In preferred forms, the step of attaching the magnetic fastener component to the first layer of material involves forming at least a portion of a strap for the positioning and stabilising structure.

In preferred forms, forming the portion of the strap involves laminating at least two layers of material to each other to form a multi-layer structure.

In preferred forms, the method includes the step of shaping the strap to create a desired shape. For instance, the method may include cutting e.g. die cutting or ultrasonic welding which removes material from the first layer of material.

In preferred forms, the method may include the step of attaching the strap to another component of the positioning and stabilising structure. For instance, the strap may be sewn to another component of the positioning and stabilising structure to create a joint.

One form of the present technology relates to a positioning and stabilising structure for a patient interface system, comprising
a rear strap assembly,
at least one strap which in use extends away from the rear strap assembly and along a side of the patient's face, wherein the at least one strap has a distal end, and
a first strap fastener that comprises a magnetic fastener component provided to the at least one strap and that is positioned between the distal end and the rear strap assembly.

In preferred forms, the positioning and stabilising structure comprises at least one strap and a strap fastener half, wherein the strap fastener half comprises a magnetic fastener component which is formed to the at least one strap.

In preferred forms, the positioning and stabilising structure comprises at least one strap and a strap fastener half which is permanently attached to the positioning and stabilising structure, wherein the strap fastener half comprises a magnetic fastener component, and further wherein the strap fastener half is configured to in use engage with a corresponding fastener half on a patient interface to attach the positioning and stabilising structure to the patient interface.

In preferred forms, the positioning and stabilising structure comprises a first strap and a second strap, and wherein the first strap comprises a first strap fastener half and the second strap comprises a second strap fastener half, and further wherein the first strap fastener half and the second strap fastener half each comprises a magnetic fastener component.

In preferred forms, the magnetic fastener components are both permanently attached to, or formed to, the respective strap.

In preferred forms, at least one of the first strap fastener half and the second strap fastener half comprises an insertion portion configured to in use be inserted into a corresponding receiving portion of a patient interface fattener half.

In preferred forms, the strap fastener half is attached to a first layer of material.

In preferred forms, the first layer of material is a textile material.

In preferred forms, the strap(s) have a multi-layer construction comprising the first layer of material and at least one additional layer of material.

In preferred forms, the multi-layer construction comprises at least one additional layer and wherein the additional layer of material is a textile material.

In preferred forms, the multi-layer construction comprises a layer of foam material.

In preferred forms, the multi-layer construction comprises a plurality of layers that are laminated or glued together.

In preferred forms, the strap has a patient contacting surface, and wherein the strap fastener component is located on the distal side of the patient contacting surface from the patient's face in use.

In preferred forms, the positioning and stabilising structure further comprises a third strap and a fourth strap.

In preferred forms, the first strap and the second strap provide a pair of lower straps for the positioning and stabilising structure and the third strap and the fourth strap provide a pair of upper straps for the positioning and stabilising structure.

In preferred forms, the third strap and the fourth strap each comprises a connector configured to in use releasably attach the positioning and stabilising structure to a patient interface.

In preferred forms, the connectors on the third strap and the fourth strap each comprises a magnetic fastener component configured to in use engage with a corresponding magnetic fastener component on a patient interface.

One form of the present technology relates to a treatment system, comprising
a patient interface to deliver a supply of pressurised breathable gas to one or more of a patient's airways, and
a positioning and stabilising structure;
wherein the positioning and stabilising structure comprises at least one strap having a strap fastener half, wherein the strap fastener half comprises a magnetic fastener component which is formed to the at least one strap, and
the patient interface comprises a patient interface fastener half which includes a magnetic fastener component, and further wherein
in use the magnetic fastener components together releasably attach the at least one strap to the patient interface.

One form of the present technology relates to a method of manufacturing a first fastener half of a positioning and stabilising structure for a patient interface system, the method comprising the following steps:
providing at least one layer of material; and
providing a fastener component to the at least one layer of material, the fastener component configured to in use magnetically engage with a fastener component of a second fastener half.

In preferred forms, the method further includes the step of providing at least a second layer of material relative to the at least one layer of material.

In preferred forms, the at least one layer of material comprises a layer of foam, and/or the second layer is a layer of textile material, fabric or laminate material.

In preferred forms, the step of providing the fastener component to the at least one layer of material includes attaching at least a portion of the fastener component to a surface of the at least one layer of material.

In preferred forms, the step of providing the fastener component to the at least one layer of material includes forming a recess in a surface of the at least one layer of material. In these forms, the step of providing the fastener component to the at least one layer of material includes positioning at least a portion of the fastener component in the recess formed in the at least one layer of material.

In preferred forms, the step of providing the fastener component to the at least one layer of material includes forming an aperture through the at least one layer of material. In these forms, the step of providing the fastener component to the at least one layer of material includes positioning at least a portion of the fastener component through the aperture formed in the at least one layer of material.

In preferred forms, the step of forming the recess or aperture in the at least one layer of material includes radio frequency (ultra-sonic) welding, cutting, pressing or other technique.

In some forms, the step of providing the fastener component to the at least one layer of material includes positioning at least a portion of the fastener component between the at least one layer of material and the second layer of material.

In preferred forms, the step of providing the fastener component to the at least one layer of material includes positioning the entire fastener component between the at least one layer of material and the second layer of material.

In preferred forms, the method further comprises the step of forming the first layer of material and the second layer of material together.

In preferred forms, the step of forming the at least one layer of material and the second layer of material together involves at least one of ultrasonic torsional welding, applying adhesive to one or more of the layers, heat bonding and/or adhesive potting.

In preferred forms, the step of providing the fastener component to the at least one layer of material includes attaching the fastener component to the at least one layer of material such that a portion of the fastener component protrudes away from a transverse plane of the first fastener half manufactured according to any of the forms described herein.

In preferred forms, the step of providing the fastener component to the at least one layer of material includes attaching the fastener component to the at least one layer of material such that a portion of the fastener component at least partially extends through the at least one layer of material.

In preferred forms, the step of providing a fastener component to the at least one layer of material includes providing a second fastener component to the at least one layer of material.

In preferred forms, the step of providing the fastener component to the at least one layer of material involves attaching a male component to the at least one layer of material. In these forms, the step of attaching the male component to the at least one layer of material further includes attaching a magnet to the at least one layer of material. In these forms, the step of attaching the male component to the at least one layer of material includes attaching a first and second part of the male component to opposing surfaces of the at least one layer of material such that a magnet is positioned between the first part and the opposing second part, wherein the step of attaching the first and second part of the male component includes forming an aperture through the at least one layer of material, positioning the magnet between the first part and opposing second part, attaching the first part to a first surface of the at least one layer of material and attaching the opposing second part to an opposing second surface of the at least one layer of material to secure the first and second part relative to each other to secure the magnet relative to the at least one layer of material.

In some forms, the method further includes the step of providing the first fastener half to the patient interface or the positioning and stabilising structure.

One form of the present technology relates to a first fastener half of a positioning and stabilising structure for a patient interface system, comprising:
at least one layer of material; and
a fastener component provided to the at least one layer of material, wherein the fastener component is configured to in use magnetically engage with a fastener component of a second fastener half.

In preferred forms, the fastener component comprises an insertion portion configured to in use be inserted into a corresponding receiving portion of the other fastener component.

In preferred forms, the insertion portion is configured to in use magnetically engage the corresponding receiving portion.

In preferred forms, the first fastener half comprises further fastener component(s) provided to the at least one layer of material.

In preferred forms, the fastener component is configured to in use facilitate rotational movement with respect to the other fastener component.

In preferred forms, the insertion portion is configured to in use facilitate rotational movement with respect to the receiving portion.

In preferred forms, the fastener component comprises a magnet or is configured to generate a magnetic field.

In preferred forms, the fastener component is configured to be attracted to a magnetic field. In these forms the fastener component of the second fastener half is configured to generate a magnetic field.

In preferred forms, the fastener component is spherical or cylindrically-shaped.

In preferred forms, the first fastener half be configured to physically engage and a magnetically engage with the second fastener half.

In preferred forms, the first fastener half is configured to physically engage with the second fastener half. The physical interaction may be a press fit, snap-fit, friction fit, clipping structure or other physical fastener arrangement.

In some forms, the first fastener half comprises a male component which is configured to physically engage with a complementary female component configured on the second fastener half. The male component secures the magnet relative to the at least one layer of material. The male component forms the insertion portion. In preferred forms, the male component comprises a first part and an opposing second part configured to attach to opposing surfaces of the at least one layer of material.

In preferred forms, the fastener component comprises a spherical, cylindrical or any other suitably shaped member configured to be attracted by a magnetic field.

In preferred forms, the fastener component is provided a portion of a headgear strap for the positioning and stabilising structure. In these forms, the first fastener half forms a part of the headgear strap.

In preferred forms, the fastener component is provided to a support structure. In some of these forms, the support structure may be adapted to receive a portion of a headgear strap. In others, the support structure may be provided to the patient interface.

In preferred forms, the first fastener half comprising the fastener component is manufactured by the method according to any of the forms described herein.

One form of the present technology relates to a method of manufacturing a second fastener half of a positioning and stabilising structure for a patient interface system, the method comprising the following steps:
providing at least one layer of material; and
providing a fastener component to the at least one layer of material, wherein the fastener component is configured to in use magnetically engage with a fastener component of a first fastener half.

In preferred forms, the step of providing at least one layer of material includes providing at least one layer of foam, textile material, fabric and/or laminate material.

In some forms, the method further includes the step of providing the at least one layer of material to the patient interface system or the positioning and stabilising structure.

In preferred forms, the step of providing the fastener component to the at least one layer of material includes attaching the fastener component to the at least one layer of material. This step involves at least one of welding, bonding, adhering, or otherwise attaching the fastener component to the at least one layer of material.

In preferred forms, the step of providing the fastener component to the at least one layer of material includes attaching an eyelet, washer or a female component comprising a receiving structure to the at least one layer of material. In these forms, the step of providing the fastener component to the at least one layer of material includes forming an aperture through the at least one layer of material, and attaching the eyelet, washer or the female component to the at least one layer of material about the aperture. In these forms, the step of providing the eyelet or female component to the at least one layer of material includes attaching a first part to a first surface of the at least one layer of material about the aperture and attaching an opposing second part to an opposing second surface of the at least one layer of material about the aperture to secure the first and second part relative to each other and relative to the at least one layer of material.

One form of the present technology relates to a second fastener half of a positioning and stabilising structure for a patient interface system, comprising:
at least one layer of material; and
a fastener component provided to the at least one layer of material, wherein the fastener component is configured to in use magnetically engage with a fastener component of a first fastener half.

In preferred forms, the fastener component is the fastener component of the first fastener half according to any of the forms described herein.

In preferred forms, the at least one layer of material comprises at least one layer of foam, textile material, fabric and/or laminate material.

In preferred forms, the fastener component comprises a receiving portion configured to in use receive the corresponding insertion portion of the first fastener half. In these forms, the receiving portion comprises a cavity configured to receive the insertion portion.

In preferred forms, the fastener component is configured to in use facilitate rotational movement with respect to the fastener component of the first fastener half.

In preferred forms, the receiving portion is configured to in use facilitate rotational movement with respect to the insertion portion of the fastener component.

In some forms, the fastener component comprises a magnet or is configured to generate a magnetic field.

In preferred forms, the fastener component is configured to be attracted by a magnetic field. In these forms the fastener component of the first fastener half is configured to generate a magnetic field.

In some forms, the fastener component comprises a hollow magnet.

In preferred forms, the fastener component comprises an eyelet or washer configured to be attracted by a magnetic field or a female component configured to be attracted by a magnetic field.

In preferred forms, the eyelet or female component comprise a first part and an opposing second part configured to attach to opposing surfaces of the at least one layer of material.

In preferred forms, the second fastener half, the fastener component is provided to a portion of a headgear strap for the positioning and stabilising structure. In these forms, the second fastener half forms a part of the headgear strap.

In preferred forms, the fastener component is provided to a support structure. In some of these forms, the support structure may be adapted to receive a portion of a headgear strap. In others, the support structure may be provided to the patient interface.

In preferred forms, the second fastener half is manufactured by the method according to any of the forms described herein.

It should be understood that one or more of the steps in the method may be performed substantially concurrently with, or prior to completion of, another step of the method. Therefore, the steps described herein should not be considered as discreet from each other.

Throughout the specification, reference to the term “magnetic fastener component” should be understood to mean a part or assembly which generates or which can interact with a magnetic field. In some embodiments, a magnetic component may be a permanent magnet. Alternatively, in other embodiments, a magnetic component may have material properties that are influenced by e.g. attracted or repelled by, a magnetic field; for instance, a magnetic component may be made from or include a ferro-magnetic material so that it is attracted to a magnetic field. In use, the magnetic field, or interaction with a magnetic field of another magnetic fastener component, creates a retention force to attach two components together.

It is envisaged that the present technology may use different combinations of materials to form at least one pair of fastener halves. For instance, a pair of fastener halves may be provided by a ferromagnetic component and a magnetic component, or two magnetic components.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

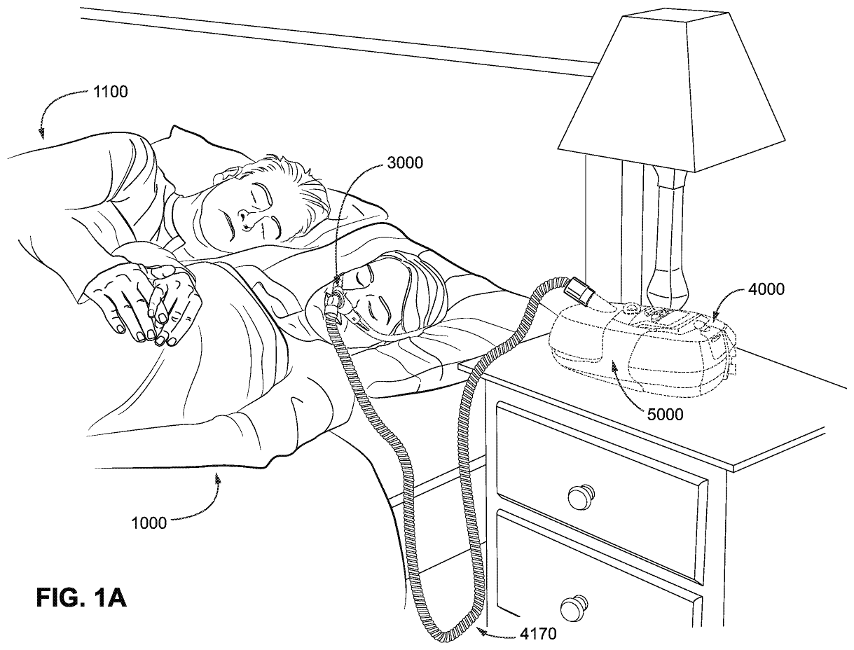

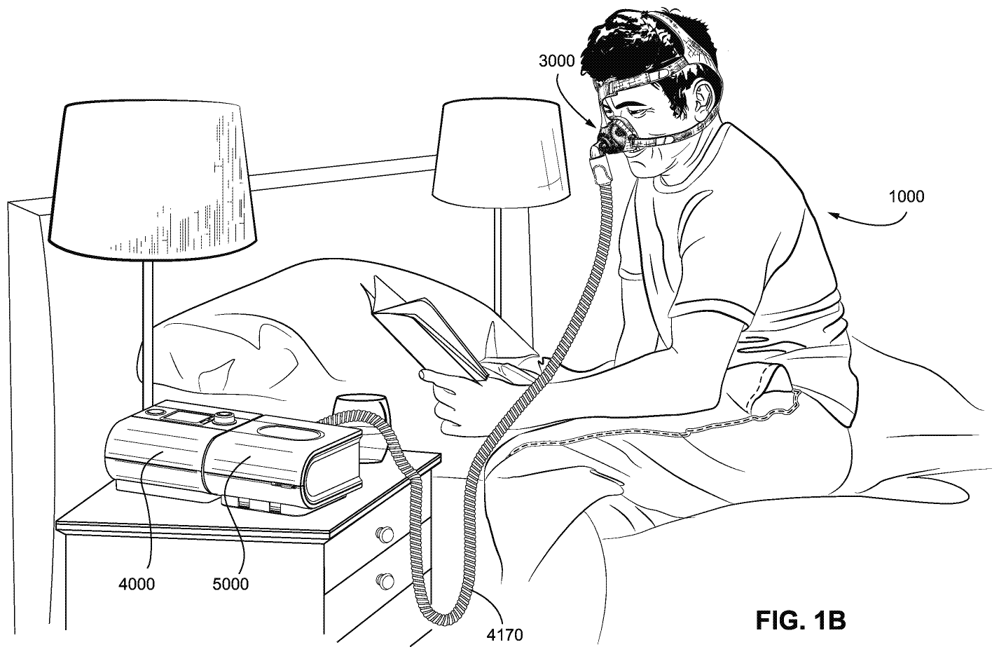

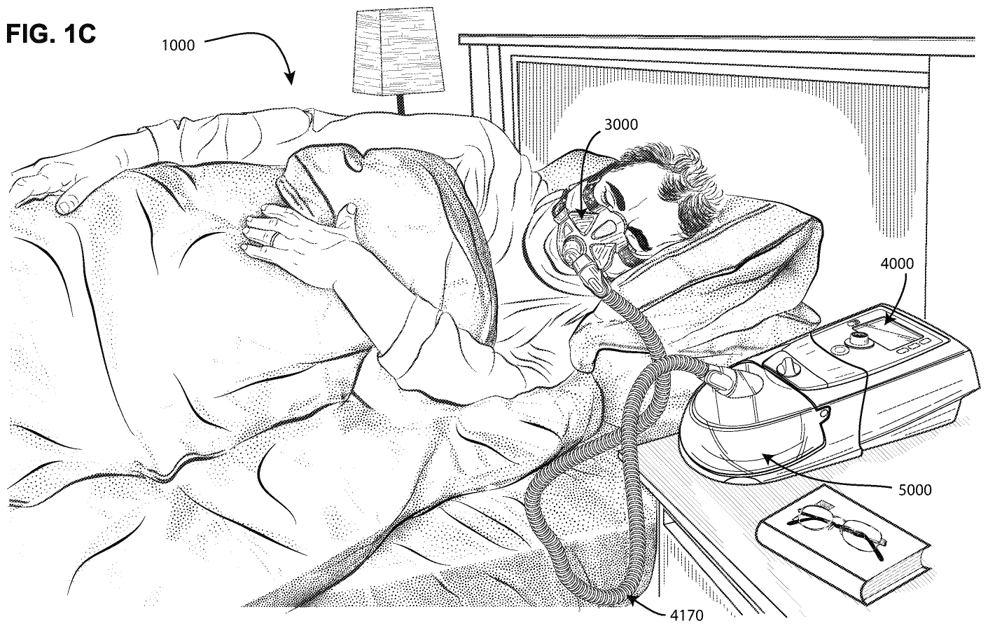

FIG. 1A shows a system including a patient 1000 wearing a patient interface 3000, in the form of nasal pillows, receiving a supply of air at positive pressure from an RPT device 4000. Air from the RPT device 4000 is conditioned in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 3.2 Respiratory System and Facial Anatomy

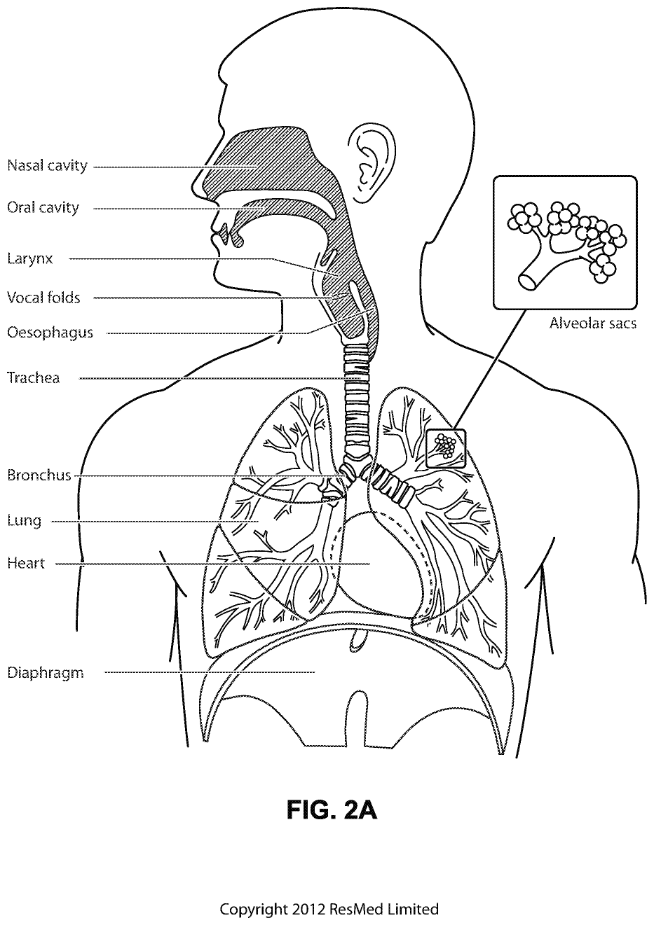

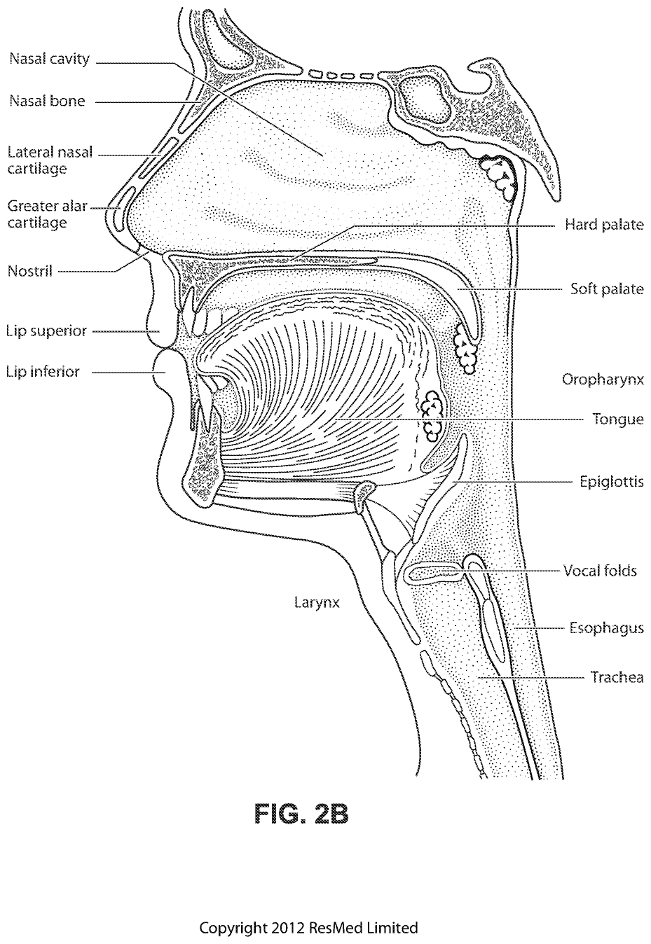

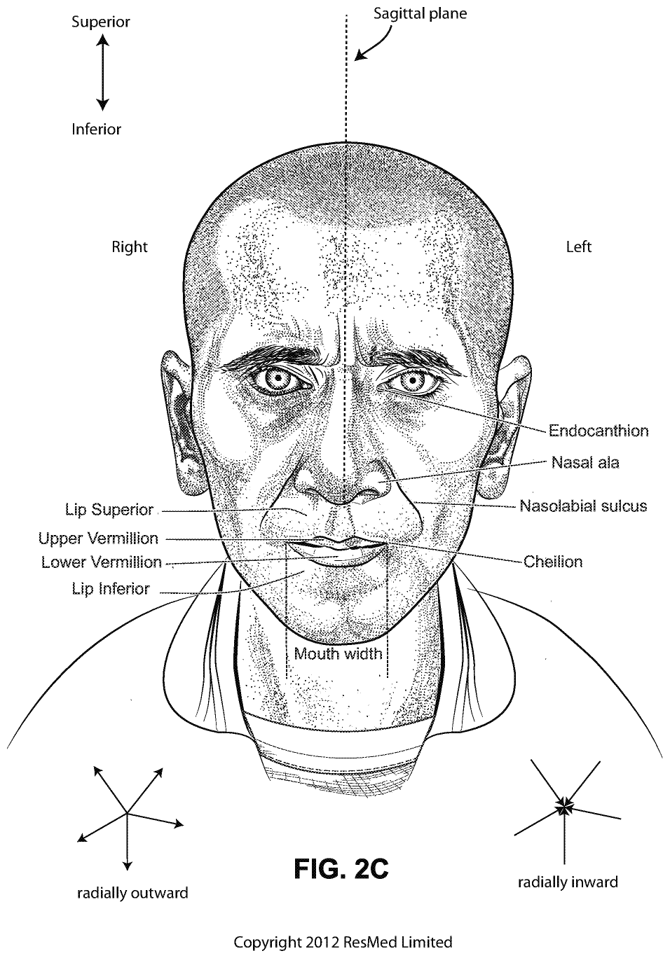

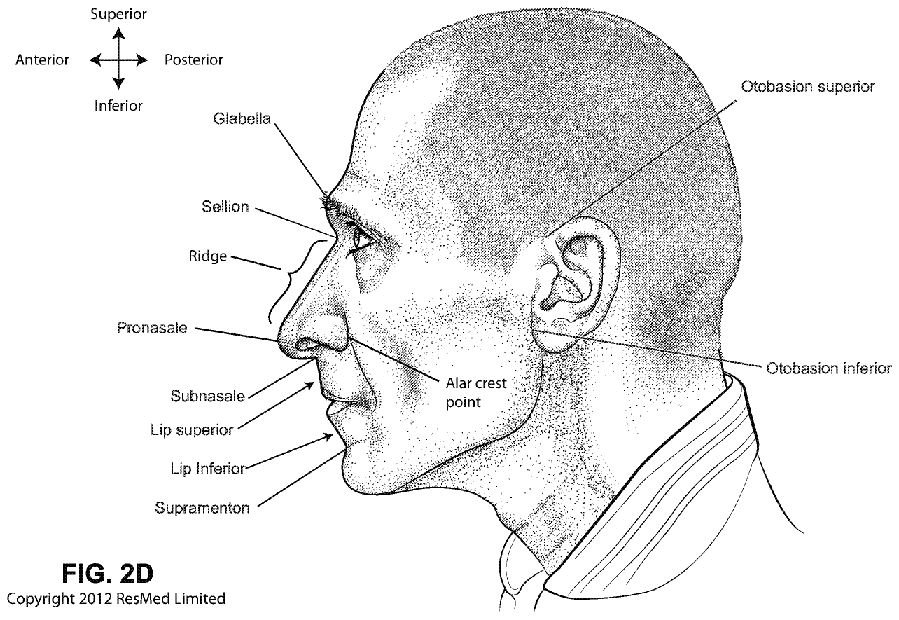

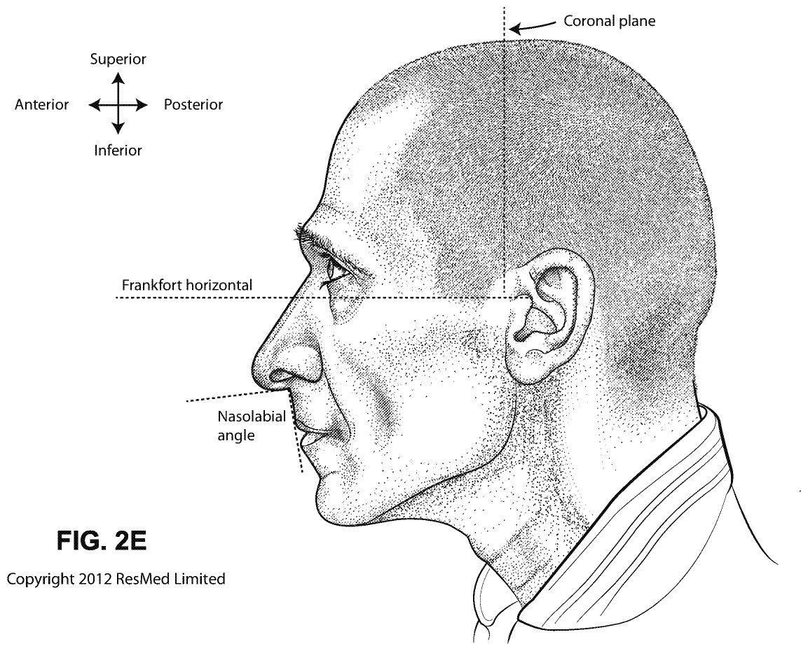

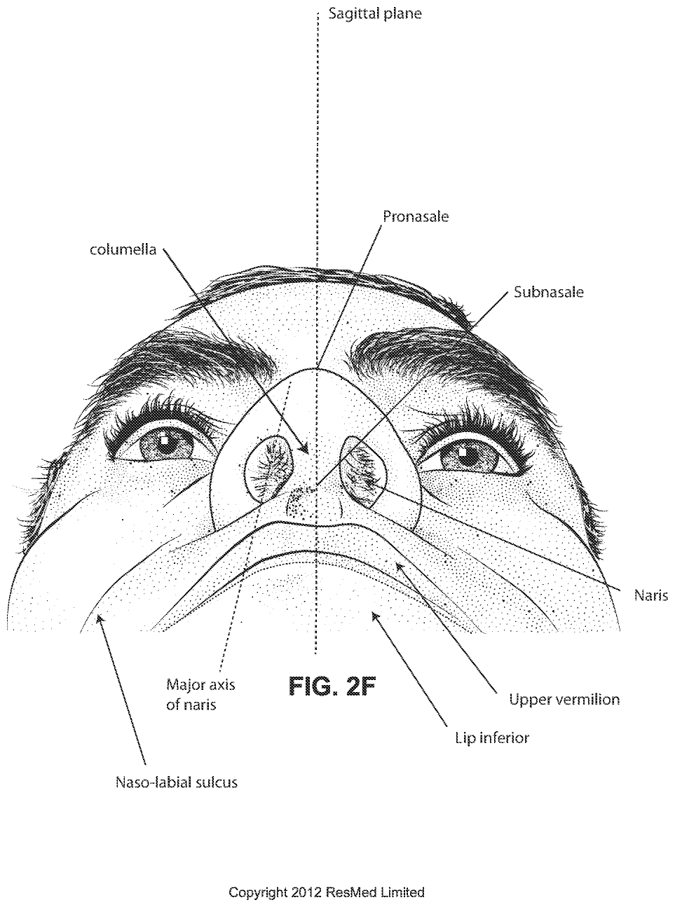

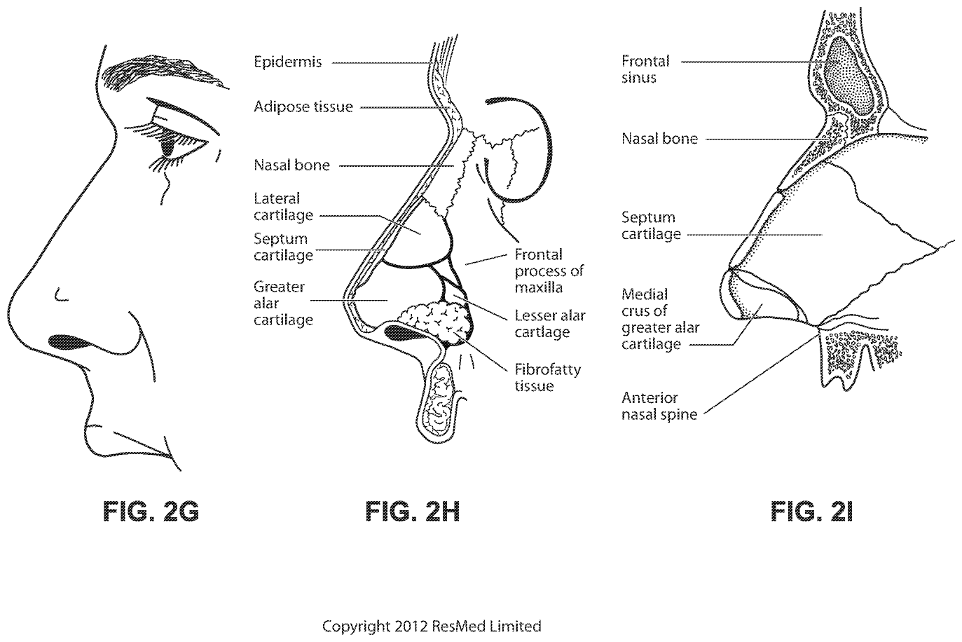

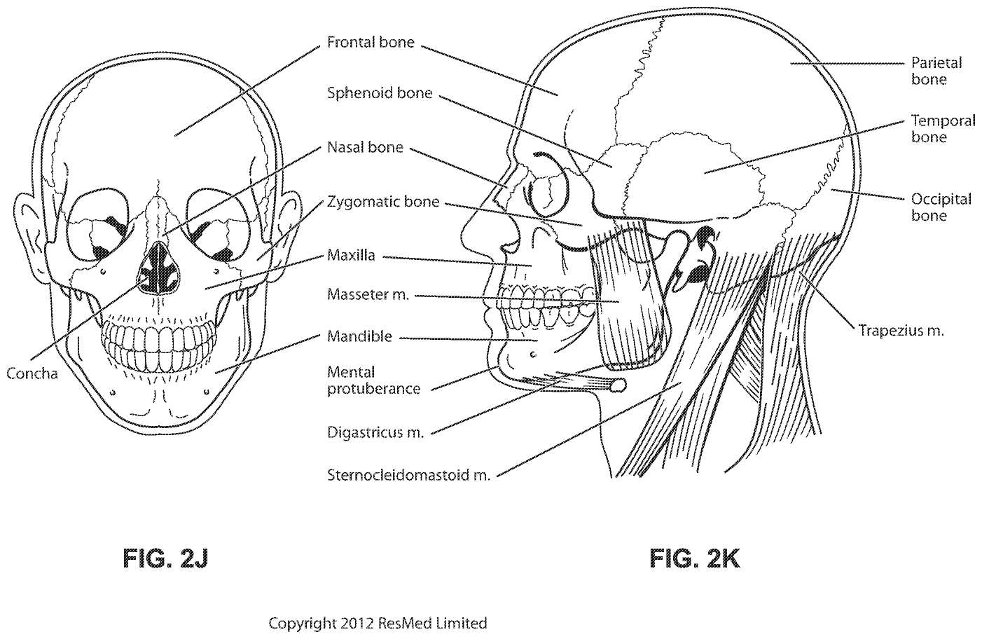

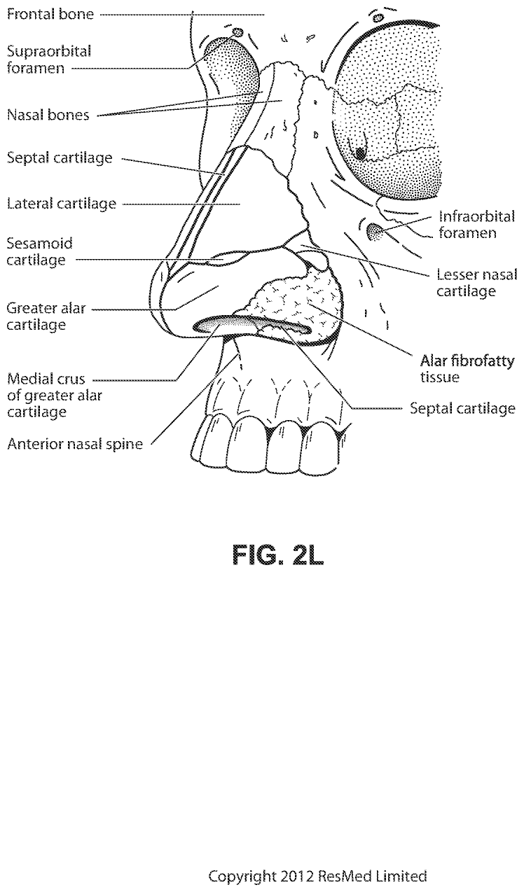

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoid and trapezius.

FIG. 2L shows an anterolateral view of a nose.

### 3.3 Patient Interface System

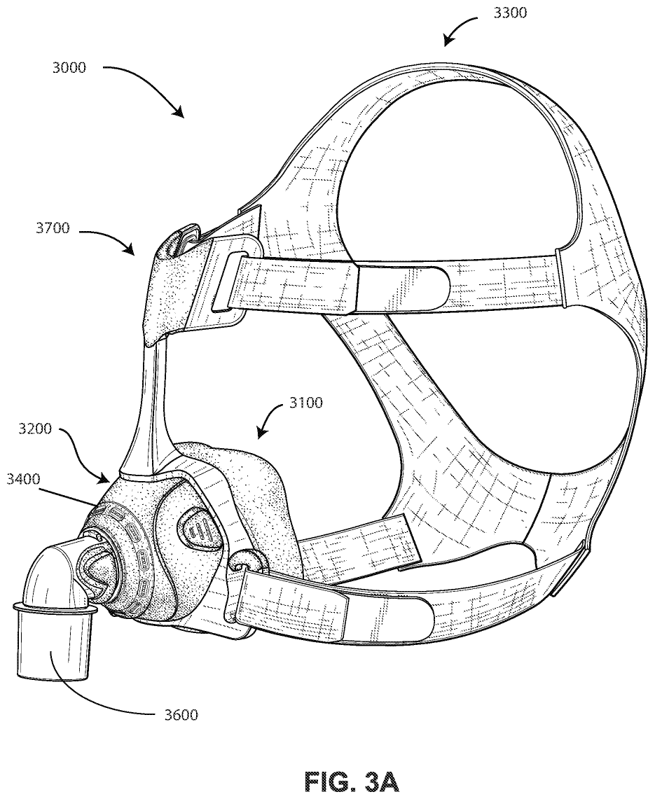

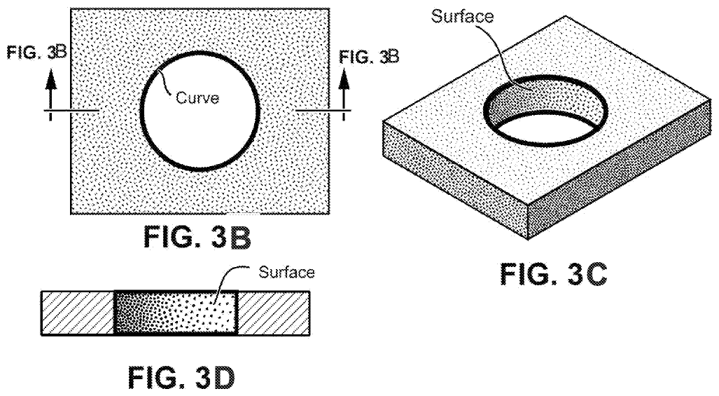

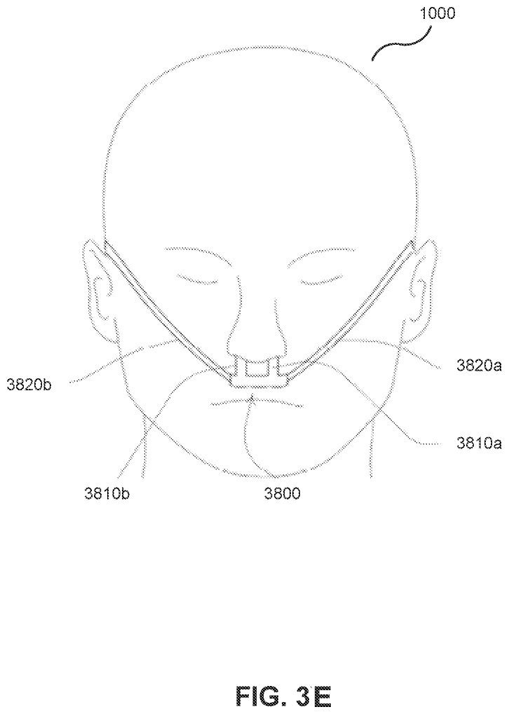

FIG. 3A shows a patient interface system in the form of a nasal mask in accordance with one form of the present technology.

FIG. 3B shows the surface of a structure, with a one dimensional hole in the surface. The illustrated plane curve forms the boundary of a one dimensional hole.

FIG. 3C shows a cross-section through the structure of FIG. 3B. The illustrated surface bounds a two dimensional hole in the structure of FIG. 3B.

FIG. 3D shows a perspective view of the structure of FIG. 3B, including the two dimensional hole and the one dimensional hole. Also shown is the surface that bounds a two dimensional hole in the structure of FIG. 3B.

FIG. 3E shows a patient interface in the form of a nasal cannula in accordance with one form of the present technology.

### 3.4 RPT Device

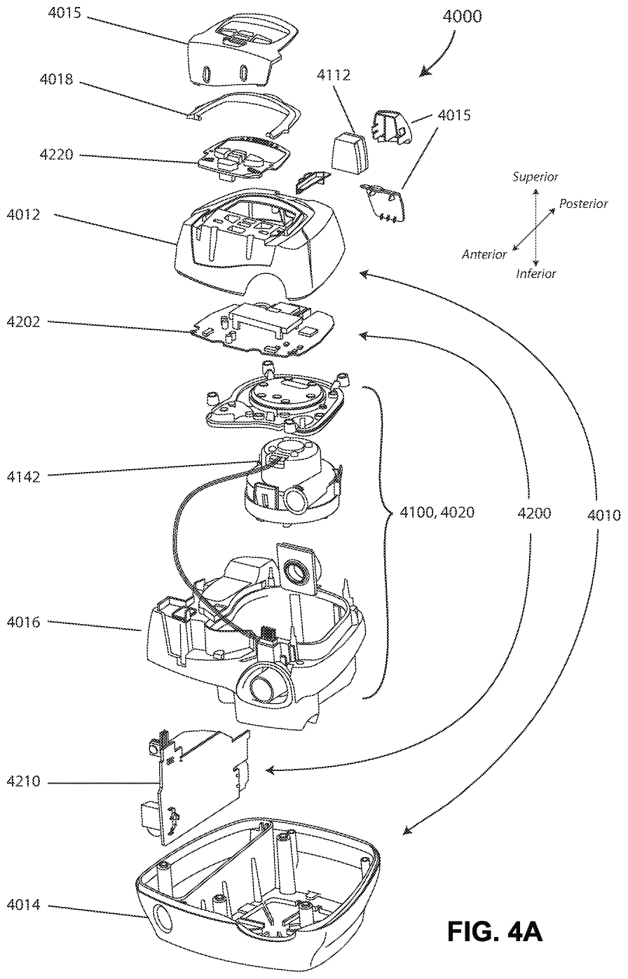

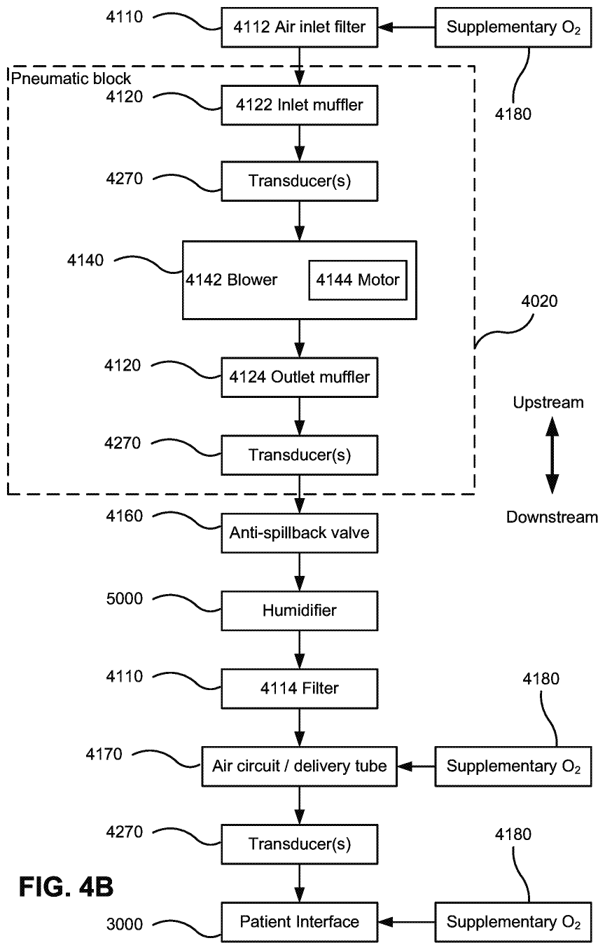

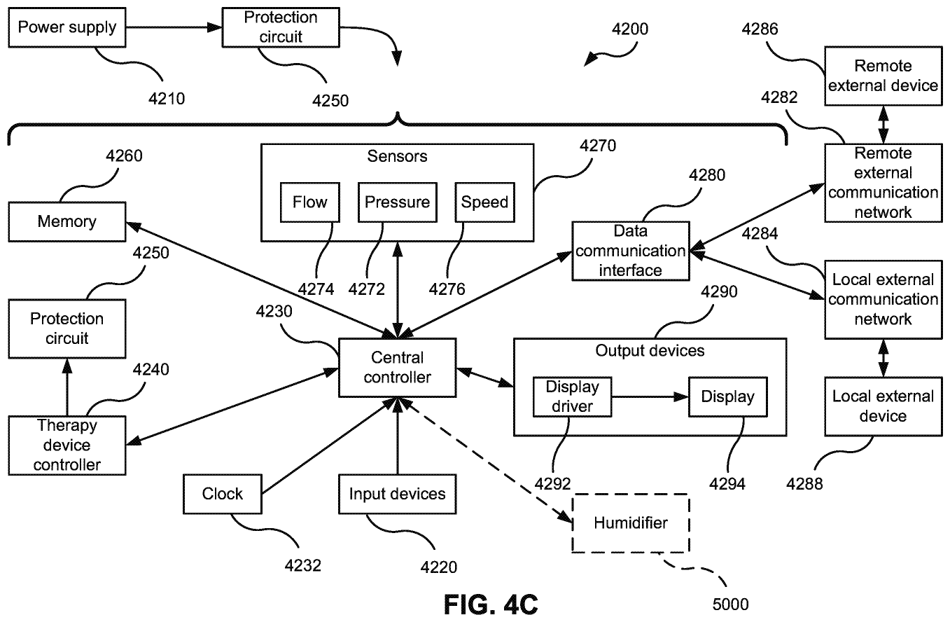

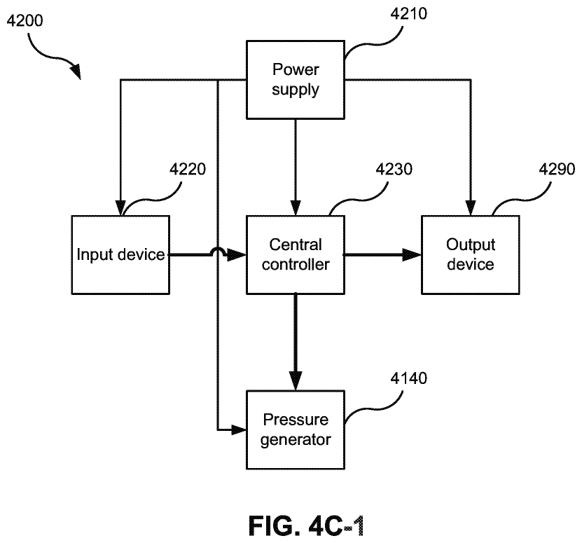

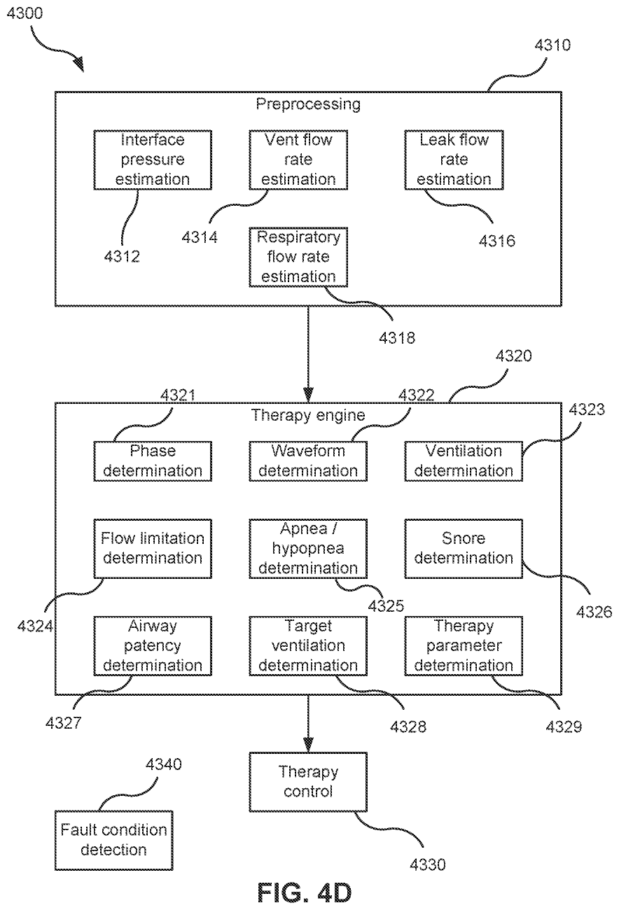

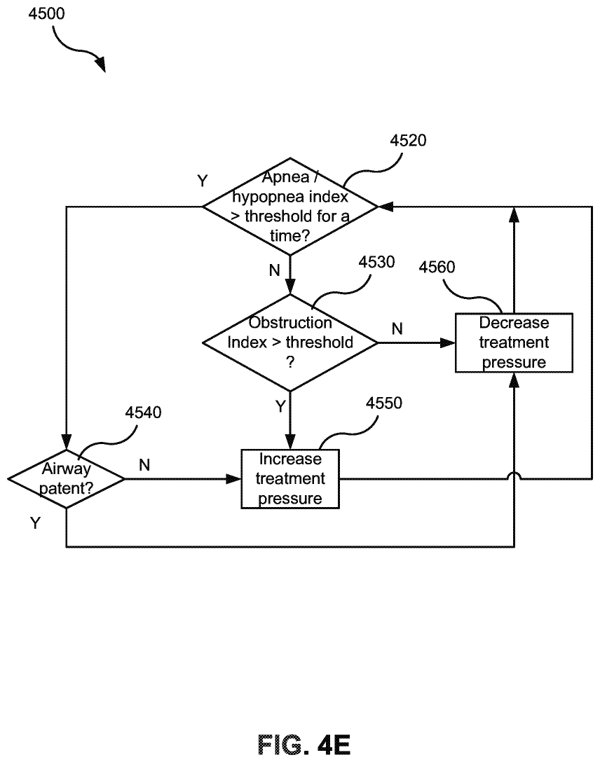

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

FIGS. 4C and 4C-1 are schematic diagrams of the electrical components of an RPT device in accordance with forms of the present technology.

FIG. 4D is a schematic diagram of the algorithms implemented in an RPT device in accordance with one form of the present technology.

FIG. 4E is a flow chart illustrating a method carried out by the therapy engine module of FIG. 4D in accordance with one form of the present technology.

### 3.5 Fasteners

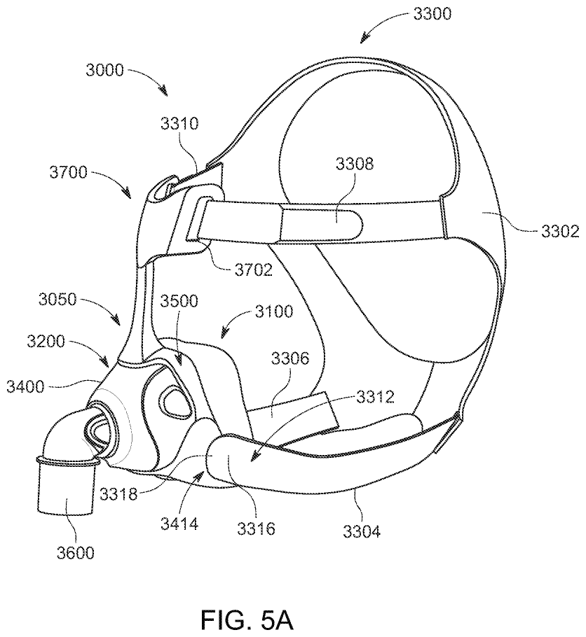

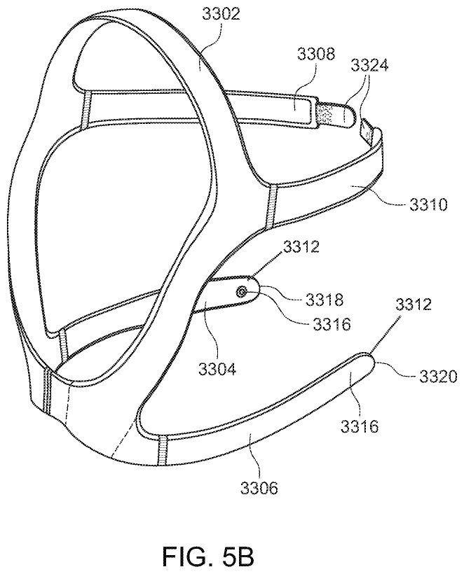

FIG. 5A is a first side view of a patient interface system including a positioning and stabilising structure in accordance with one form of the present technology.

FIG. 5B is a first side view of headgear forming part of a positioning and stabilising structure according to an aspect of the invention.

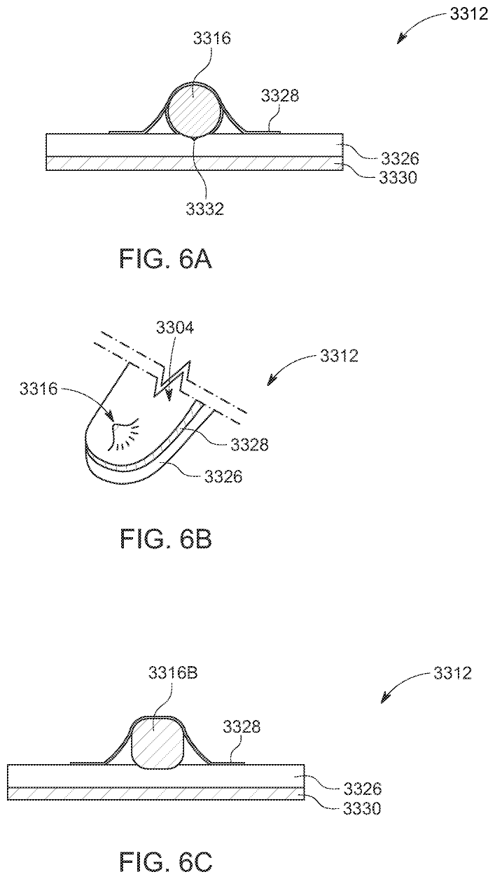

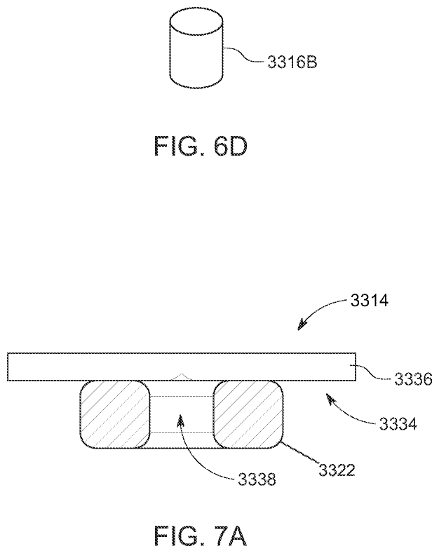

FIG. 6A is a side cross-sectional view of a first fastener component in accordance with one form of the present technology.

FIG. 6B is a perspective view of a first fastener half in accordance with another form of the present technology.

FIG. 6C is a side cross-sectional view of a first fastener half in accordance with one form of the present technology.

FIG. 6D shows a representative magnetic component for use in a fastener arrangement in accordance with one form of the present technology.

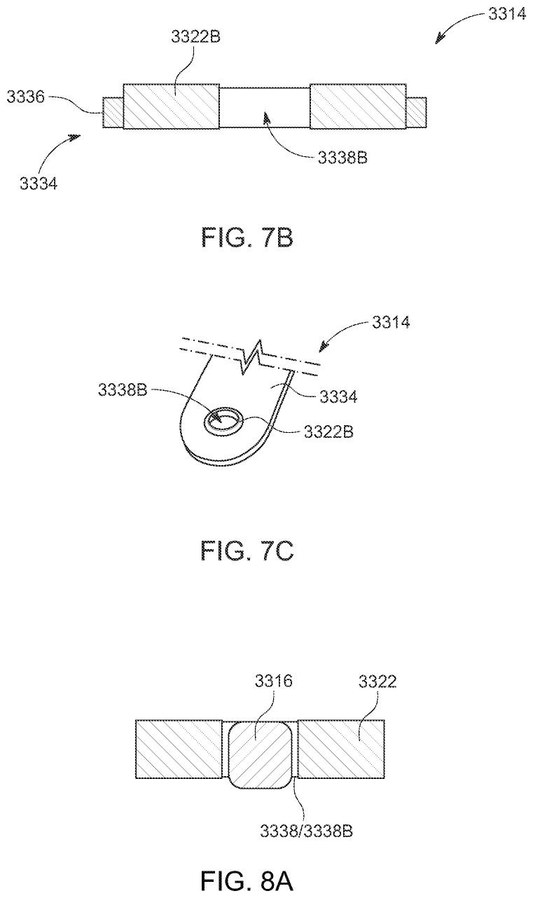

FIG. 7A is a side cross-sectional view of a second fastener half in accordance with one form of the present technology.

FIG. 7B is a side cross-sectional view of a first fastener half in accordance with another form of the present technology.

FIG. 7C is a perspective view of a second fastener half in accordance with one form of the present technology.

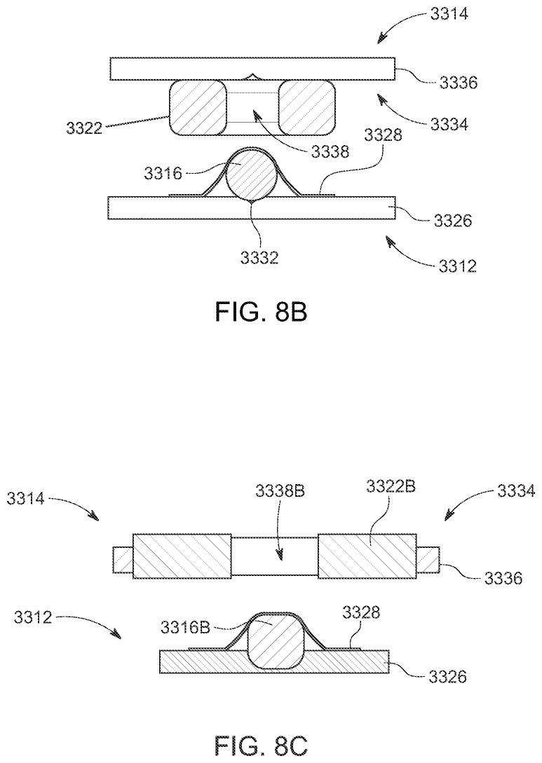

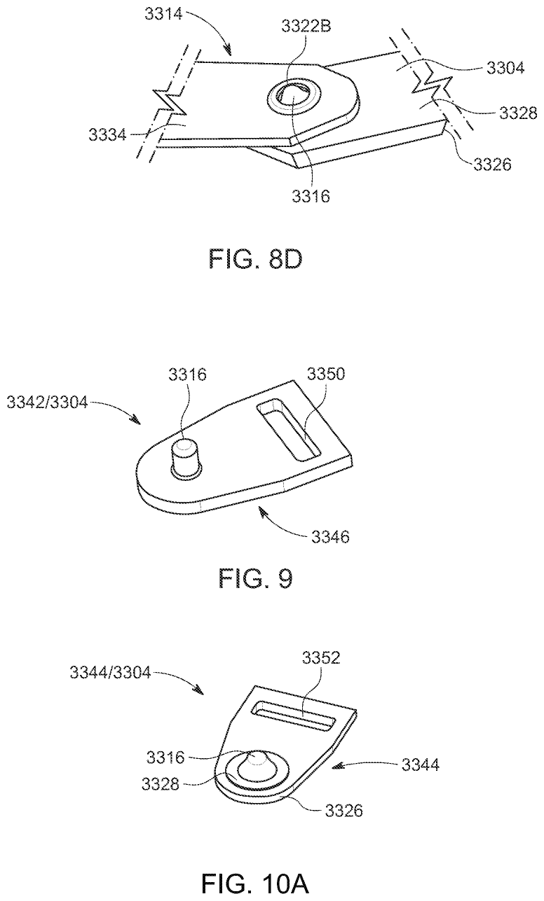

FIG. 8A is representative cross-sectional view showing the position of a first fastener half and a second fastener half of a fastener arrangement in accordance with one form of the present technology, when engaged with each other.

FIG. 8B is a side cross-sectional view of a fastener arrangement in accordance with one form of the present technology.

FIG. 8C is a side cross-sectional view of a fastener arrangement in accordance with another form of the present technology.

FIG. 8D is a perspective view of a fastener arrangement in accordance with another form of the present technology.

FIG. 9 is a perspective view of a first fastener half in accordance with another form of the present technology.

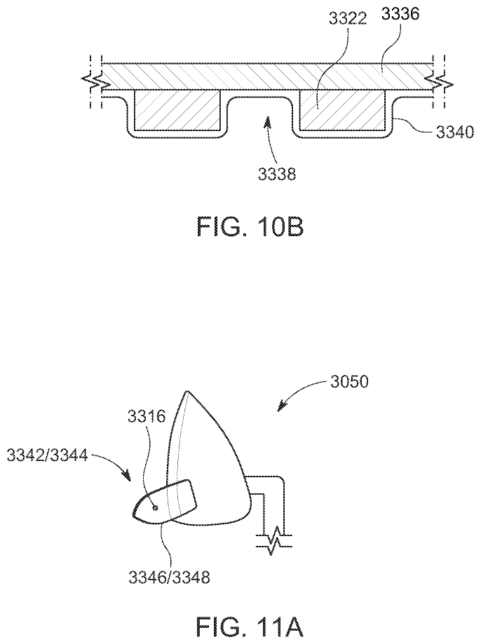

FIG. 10A is a perspective view of a first fastener half having a covered magnetic fastener component in accordance with one form of the present technology.

FIG. 10B is a side cross-sectional view of a second fastener half in accordance with another form of the present technology.

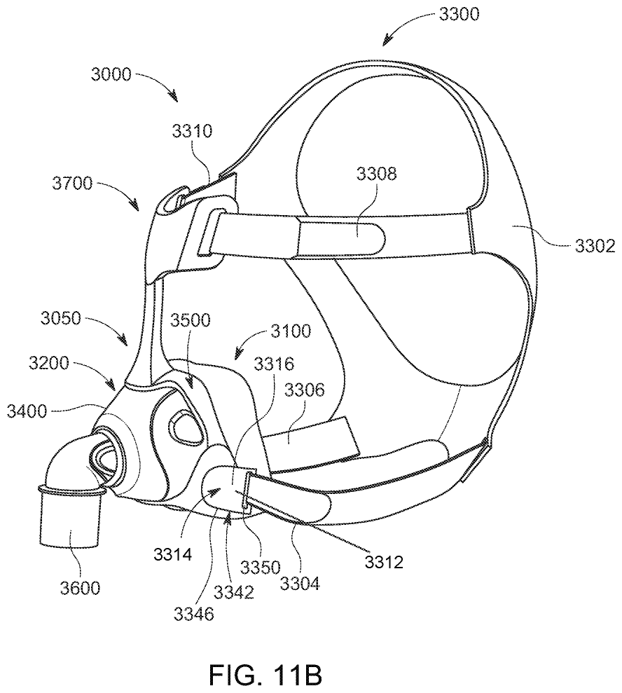

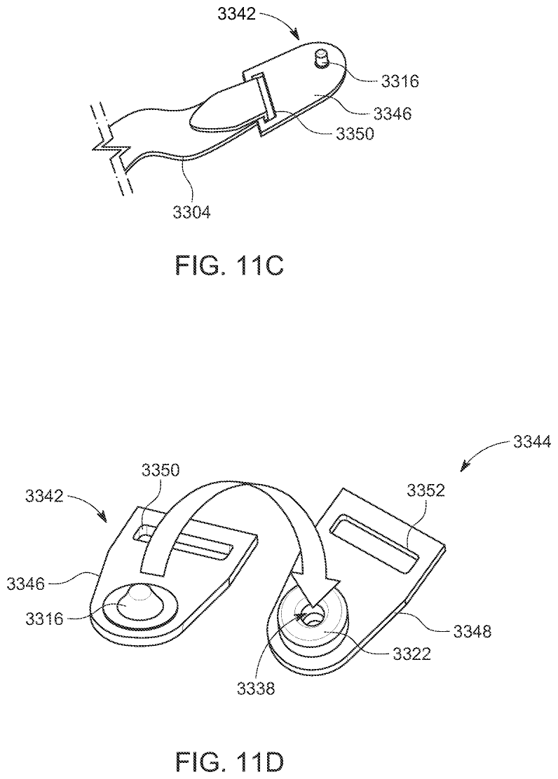

FIG. 11A is a first side view of a patient interface showing a fastener half provided to a lateral side of the patient interface in accordance with one form of the present technology.

FIG. 11B is a first side view of a patient interface system having a fastener arrangement in accordance with one form of the present technology.

FIG. 11C is an enlarged view of the first fastener half provided to a headgear strap as shown in FIG. 11B.

FIG. 11D shows how two components of a fastener arrangement according to one form of the present technology may engage each other.

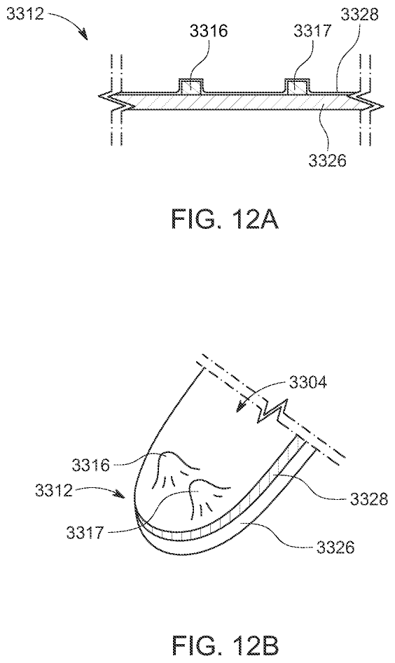

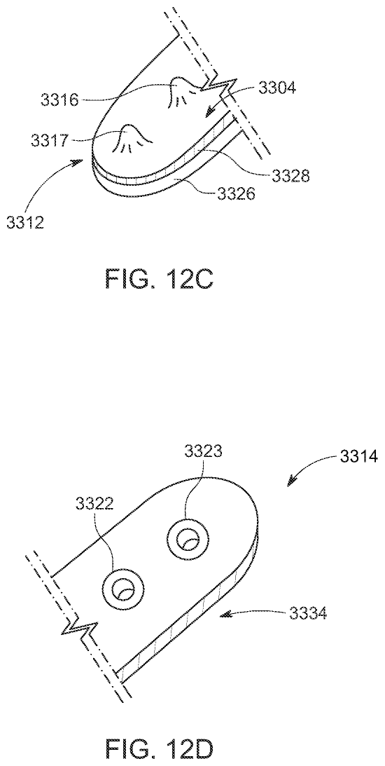

FIG. 12A is a side cross-sectional view of a first fastener half comprising two magnetic fastener components in accordance with one form of the present technology.

FIG. 12B is a perspective view of the first fastener half shown in FIG. 12A.

FIG. 12C is a perspective view of first fastener half comprising two magnetic fastener components in accordance with another form of the present technology.

FIG. 12D is a perspective view of a second fastener half comprising two magnetic fastener components in accordance with another form of the present technology.

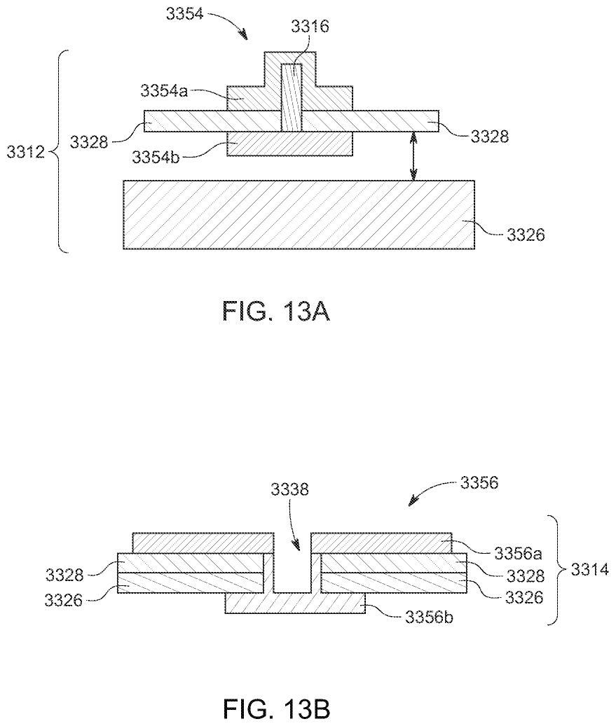

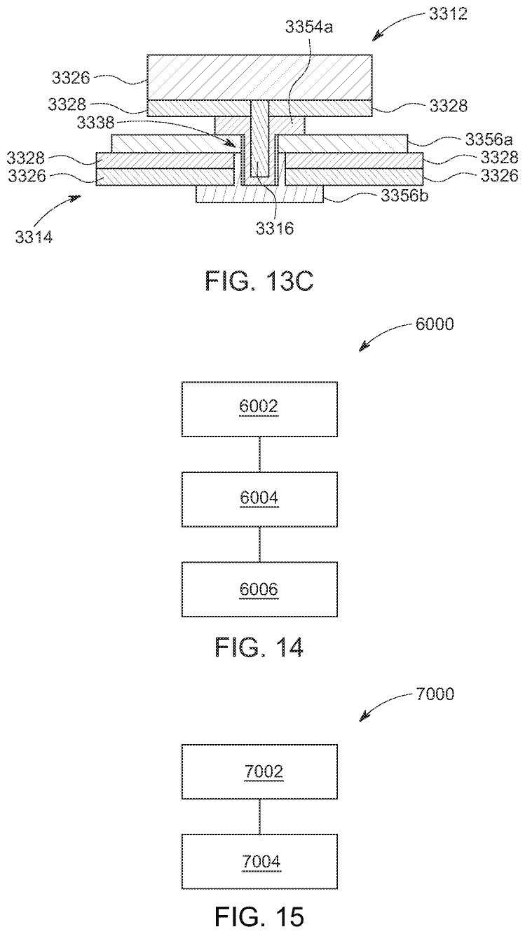

FIG. 13A is a side cross-sectional view of a first fastener half in accordance with another form of the present technology.

FIG. 13B is a side cross-sectional view of a second fastener half in accordance with another form of the present technology.

FIG. 13C is a side cross-sectional view of a fastener arrangement showing the first fastener half of FIG. 13A and the second fastener half of FIG. 13B, when engaged with each other.

### 3.6 Methods of Manufacture

FIG. 14 is a flow chart of representative steps in a method of manufacturing a positioning and stabilising structure according to the present technology.

FIG. 15 is a flow chart of representative steps in a method of manufacturing a first fastener half of a positioning and stabilising structure comprising a fastener component according to the present technology.

### 3.7 Conduit Headgear Patient Interface System

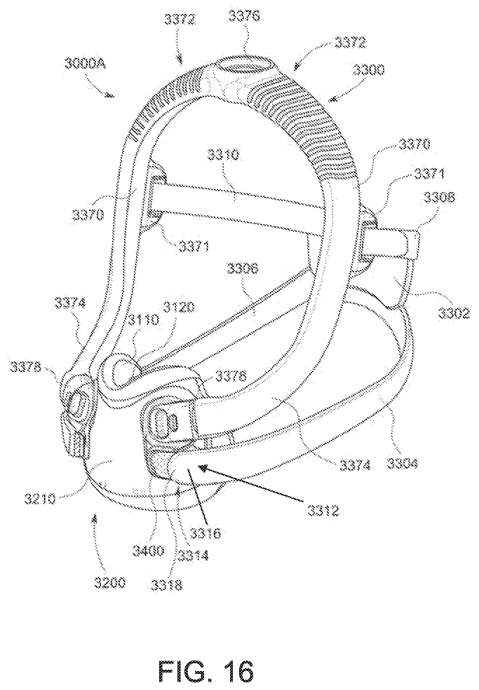

FIG. 16 is a first side view of a patient interface system including a positioning and stabilising structure in accordance with one form of the present technology.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface system 3000 or 3800.

### 4.3 Patient Interface System

A non-invasive patient interface system 3000 in accordance with one aspect of the present technology comprises the following functional aspects: a patient interface 3050 having a seal-forming structure 3100 and a plenum chamber 3200, a positioning and stabilising structure 3300, a vent 3400, one form of connection port 3600 for connection to air circuit 4170. The patient interface system 3000 may also comprise a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface system 3000 is therefore suitable for delivery of positive pressure therapy.

An unsealed patient interface system 3800, in the form of a nasal cannula, includes nasal prongs 3810a, 3810b which can deliver air to respective nares of the patient 1000 via respective orifices in their tips. Such nasal prongs do not generally form a seal with the inner or outer skin surface of the nares. The air to the nasal prongs may be delivered by one or more air supply lumens 3820a, 3820b that are coupled with the nasal cannula 3800. The lumens 3820a, 3820b lead from the nasal cannula 3800 to a respiratory therapy device via an air circuit. The unsealed patient interface system 3800 is particularly suitable for delivery of flow therapies, in which the RPT device generates the flow of air at controlled flow rates rather than controlled pressures. The “vent” at the unsealed patient interface system 3800, through which excess airflow escapes to ambient, is the passage between the end of the prongs 3810a and 3810b of the cannula 3800 via the patient's nares to atmosphere.

If a patient interface system 3000, 3800 is unable to comfortably deliver a minimum level of positive pressure to the airways, the patient interface may be unsuitable for respiratory pressure therapy.

The patient interface system 3000, 3800 in accordance with one form of the present technology is constructed and arranged to be able to provide a supply of air at a positive pressure of at least 6 cmH₂O with respect to ambient.

The patient interface system 3000, 3800 in accordance with one form of the present technology is constructed and arranged to be able to provide a supply of air at a positive pressure of at least 10 cmH₂O with respect to ambient.

The patient interface system 3000, 3800 in accordance with one form of the present technology is constructed and arranged to be able to provide a supply of air at a positive pressure of at least 20 cmH₂O with respect to ambient.

### 4.3.1 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface 3050 was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

### 4.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 4.3.1.2 Nose Bridge or Nose Ridge Region

In one form, the non-invasive patient interface system 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 4.3.1.3 Upper Lip Region

In one form, the non-invasive patient interface system 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 4.3.1.4 Chin-Region

In one form the non-invasive patient interface system 3000 comprises a seal-forming structure that forms a seal in use on a chin-region of the patient's face.

In one form, the seal-forming structure includes a saddle-shaped region constructed to form a seal in use on a chin-region of the patient's face.

### 4.3.1.5 Forehead Region

In one form, the seal-forming structure that forms a seal in use on a forehead region of the patient's face. In such a form, the plenum chamber may cover the eyes in use.

### 4.3.1.6 Nasal Pillows

In one form the seal-forming structure of the non-invasive patient interface system 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 4.3.2 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

### 4.3.3 Positioning and Stabilising Structure

The seal-forming structure 3100 of the patient interface system 3000 of the present technology may be held in sealing position in use by the positioning and stabilising structure 3300.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3050.

In one form the positioning and stabilising structure 3300 provides a retention force as a safety margin to overcome the potential effect of disrupting forces on the patient interface 3050, such as from tube drag, or accidental interference with the patient interface 3050.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure 3300 comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure 3300 includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure 3300 includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilising structure 3300, each being configured to provide a retaining force to correspond to a different size and/or shape range. For example, the system may comprise one form of positioning and stabilising structure 3300 suitable for a large sized head, but not a small sized head, and another. suitable for a small sized head, but not a large sized head.

Referring now to FIG. 5A which shows additional aspects of a patient interface system 3000 in accordance with one form of the present technology.

In the embodiment of FIG. 5A, the patient interface system 3000 is shown in the form of nasal-only patient interface 3050, having a seal forming structure 3100 configured to create a seal with or around a patient's nose. However, the patient interface 3050 could be a full face mask (oro-nasal), an ultra-compact full face mask, comprise a pair of nasal pillows, a nasal cannula or a nasal cradle as should be known to one skilled in the art.

The positioning and stabilising structure 3300 has a rear strap assembly e.g. a crown strap assembly indicated generally as 3302. As illustrated, the positioning and stabilising structure 3300 has a first pair of headgear straps 3304, 3306 and a second pair of headgear straps 3308, 3310. The headgear straps 3304, 3306, 3308, 3310 in use attach the positioning and stabilising structure 3300 to the patient interface 3050. However, the positioning and stabilising structure 3300 could have a strap assembly comprising any number and/or arrangement of headgear straps as should be known to one skilled in the art.

In certain forms of the present technology, the positioning and stabilising structure 3300 is configured to releasably attach to the patient interface 3050. Therefore, the positioning and stabilising structure 3300 and the patient interface 3050 are provided with at least one pair of complementary fastener halves, e.g. the positioning and stabilising structure 3300 includes a first fastener half indicated in FIG. 5A generally as 3312 and the patient interface 3050 has a second fastener half indicated generally as 3314. In such embodiments, the first fastener half 3312, is provided to a strap e.g. the headgear strap 3304, and the second fastener half 3314 is provided to the patient interface 3050. In the illustrated embodiment of FIG. 5A the second fastener half 3314 is provided on the patient interface 3050 and the first fastener half 3312 is provided by an end of the headgear strap 3304 which is attached to the patient interface 3050. Positioning and stabilising structure with magnetic fasteners

Referring now to FIG. 5B which shows headgear 3302 forming part of a positioning stabilising structure 3300 according to an aspect of the invention. In a preferred form, the headgear 3302 of FIG. 5B is configured for use with the patient interface 3050 of FIG. 5A. However, it is to be appreciated that the headgear 3302 may be configured and/or adapted for use with any other suitable patient interface system known to one skilled in art.

In the embodiment of FIG. 5B, the fastener half 3312 includes a magnetic fastener component 3316 provided to the headgear strap 3304, while the fastener half 3312 may include a magnetic fastener component 3316 provided to the headgear strap 3306. The headgear strap 3304 may be referred to as a first upper headgear strap 3304, and the headgear strap 3306 may be referred to as a second upper headgear strap 3310.

It should also be appreciated that the headgear straps 3308, 3310 may be provided with magnetic fastener components (not illustrated) so as to facilitate attachment of the positioning and stabilising structure 3300 to the patient interface 3050. The headgear strap 3308 may be referred to as a first upper headgear strap 3308, and the headgear strap 3310 may be referred to as a second upper headgear strap 3310.

As illustrated, the first fastener half 3312 may be provided to the first lower headgear strap 3304 at or towards distal end 3318 of the first lower headgear strap 3304 which is distal to the rear strap assembly 3302. Similarly, the second fastener half 3314 may be provided at or towards distal end 3320 of the second lower headgear strap 3306 which is distal to the rear strap assembly 3302.

The positioning and stabilising structure 3300 also includes a frame 3500, and the headgear 3302 is configured to releasably attach to the frame 3500 as is illustrated in FIG. 5A. The patient interface 3050 attaches to the frame 3500 to enable the positioning and stabilising structure 3300 to hold the patient interface 3050 in position relative to a patient's airways to facilitate provision of respiratory therapy.

However, it is also envisaged that the headgear 3302 could attach directly to a patient interface 3050 e.g. the fastener halves 3312, 3314 are provided to the plenum chamber 3200 or seal forming structure 3100.

The frame 3500 includes a pair of fastener halves 3314 which are configured to in use engage with the fastener halves 3312. This facilitates attachment of the headgear 3302 to the frame 3500. Although not visible in FIG. 5A, the pair of fastener halves 3314 include magnetic fastener components 3322 provided to the frame 3500.

It should be appreciated that there are various arrangements and combinations for the magnetic fastener components 3316, 3322 and how these attach the first lower headgear strap 3304 or other headgear straps and the patient interface 3050 to each other. For instance, combinations within the scope of the present technology include that:
The magnetic fastener component 3316 is formed at least partially from a magnetic material and the magnetic fastener component 3322 is formed at least partially from a magnetic material;
The magnetic fastener component 3316 is formed at least partially from a material that is attracted to a magnetic field and the magnetic fastener component 3322 is formed at least partially from a magnetic material;
The magnetic fastener component 3316 is formed at least partially from a magnetic material and the magnetic component 3322 is formed at least partially from a material that is attracted to a magnetic field.
The magnetic fastener component(s) 3316, 3322 may be formed from a magnetic material which has an inherent magnetic field, or from a material which is attracted to a magnetic field.

The configuration and structure of the magnetic fastener components 3316, 3024 are discussed in more detail below.

It can also be seen in FIG. 5A that the frame 3500 includes a pair of fastener halves, e.g. slots 3702 which are configured to receive a respective one of the upper headgear straps 3308, 3310. As illustrated, the slots 3702 are provided in the forehead support 3700, but may also be provided lower down on the frame 3500 e.g. closer to the fastener halves 3314.

The headgear 3302 includes a strip of fastener material 3324 e.g. hooks, which are configured to in use attach to an outer surface of the first upper strap 3308 and the second upper strap 3310. This secures the upper straps 3308, 3310 in the slots 3702.

Referring now to FIG. 6A which shows a cross sectional view of a headgear strap which provides a magnetic fastener component 3316 to the headgear 3302. The headgear strap is preferably a lower headgear strap 3304, 3306 of the headgear 3302 illustrated in FIG. 5B. However, the headgear strap 3308, 3310 could also be an upper headgear strap 3308, 3310.

It can be seen that a magnetic fastener component 3316 is provided to the headgear strap 3304.

As illustrated in FIG. 6A, the first lower headgear strap 3304 has a multilayer construction including a first layer of material 3326 and at least one additional layer of material 3328. The additional layer of material 3328 may include a layer of textile material 3328 and the first layer of material 3326 may include a layer of foam material 3326.

The magnetic fastener component 3316 is positioned between the first layer of material 3326 and the additional layer of material 3328. For instance, the first layer of material 3326 and the additional layer of material 3328 may be laminated, welded, bonded, adhered, or otherwise attached to each other with the magnetic fastener component 3316 positioned therebetween.

In addition, another layer of material 3330 may be included in the multilayer structure for the headgear strap 3304. For instance, layer of material 3330 may be a layer of textile material e.g. be the same material as the first layer of material 3328. Alternatively, layer of material 3330 may be a non-textile material or other suitable material.

In some forms, the first layer of material 3326 includes a preformed recess 3332 as is partially visible in FIG. 6A and FIG. 6C. The preformed recess 3332 is sized and dimensioned to receive a portion of the magnetic fastener component 3316. This can assist with providing the magnetic fastener component 3316 in a desired position relative to the first layer of material 3326 and assist with manufacturability of the headgear strap 3304 (as is discussed in more detail below).

Referring now to FIG. 6B, the magnetic fastener component 3316 provides a raised or protruding part of the headgear strap 3304, 3306 which in use acts as an insertion portion 3316A which is configured to be inserted into a corresponding component of the magnetic fastener component 3322 (as will be discussed in more detail below).

It should be understood that various shapes and configurations for the magnetic fastener component 3316 are envisaged. For instance, as illustrated in FIG. 6A, the magnetic fastener component 3316 is in the form of a ball or sphere, e.g. is a steel ball or a spherical magnet. Alternatively, FIG. 6C shows a cross sectional view of a headgear strap 3304 in which the magnetic fastener component 3316B has a cylindrical shape, e.g. is a steel cylinder or a cylindrical magnet. It should of course be understood that other shapes for the magnetic fastener component are envisaged as within the scope of the present technology.

Referring now to FIG. 6D which shows a representative view of a magnetic fastener component 3316B. The magnetic fastener component 3316B has a cylindrical shape, e.g. with a height of substantially 6 mm and a diameter of substantially 5 mm.

The magnetic fastener component 3316, 3316B is preferably made from a magnetic material e.g. neodymium iron boron (ND₂Fe₁₄B₁). Other materials from which the magnetic fastener component 3316, 3316B can be partially or completely made include any ferromagnetic material or alloy e.g. steel.

### 4.3.3.1 Second Fastener Half

Referring now to FIG. 7A which shows a cross sectional view of a second fastener half 3322 according to an aspect of the present technology. As illustrated in FIG. 7A, the magnetic fastener component 3322 is provided to a support structure 3334.

In embodiments, the support structure 3334 may be provided on or part of the frame 3500. In other embodiments, the support structure 3334 may be provided on or part of the plenum chamber 3200, e.g. on an anterior portion 3210 of the plenum chamber 3200 shown in FIG. 16 which will be discussed in further detail below. For example, the support structure 3334 may be separately formed and attached to the plenum chamber 3200. Alternatively, the support structure 3334 may be provided directly on the plenum chamber 3200, e.g. the support structure 3334 may be formed by as part of the plenum chamber 3200. In other words the support structure 3334 may be integrally formed with the plenum chamber 3200.

In an embodiment, the support structure 3334 is formed from at least a first layer of material 3336. The first layer of material 3336 may be a soft, flexible and/or biocompatible material e.g. at least one of a foam material, a textile material and a combination of those materials. However, the first layer of material 3336 may be any other suitable material, e.g. it may be a substantially rigid material or substantially semi-rigid material.

As illustrated in FIG. 7A, the magnetic fastener component 3322 defines a receiving portion, e.g. in the form of a cavity 3338, which in use can receive the insertion portion 3316.

The cavity 3338 may have various shapes. In the embodiment illustrated in FIG. 7A, the cavity 3338 has a generally cylindrical shape. However, the cavity 3338 may have other shapes such as a concave recess, spherical, pyramidal or a prism shape. In yet a further embodiment, the magnetic fastener component 3322 may not have a cavity or recess.

As illustrated in FIG. 7A, the magnetic fastener component 3322 may be attached directly to an outer surface of the support structure 3334 e.g. by ultrasonic welding, adhesive, or other technique.

Alternatively, the magnetic fastener component 3322 may be an eyelet 3322B that is provided to a layer of material e.g. foam or textile material. For instance, the eyelet 3322B can be formed from two parts which clip together from opposite sides of the layer(s) of material 3336 forming the support structure 3334.

In embodiments, the eyelet 3322B can provide an aperture 3338B from one side of the layer of material to the other as is best shown in FIGS. 7B and 7C. Alternatively, the eyelet 3322B may not completely extend through the layer(s) of material 3336 but still provides a cavity 3338.

In the example shown in FIG. 7A, the magnetic fastener component 3322 comprises a magnet, e.g. a hollow magnet 3322.

In other examples, the magnetic fastener component 3322 is constructed at least partially from a material that is attracted by a magnetic field.

### 4.3.3.2 Magnetic Engagement of Fastener Halves

In forms of the present technology, the magnetic fastener components 3316, 3322 are configured to magnetically engage with each other in use. To facilitate this, the magnetic fastener components 3316, 3322 are positioned sufficiently close to each other to allow the respective magnetic field(s) to interact.

In preferred embodiments, the magnetic fastener component 3316, 3316B can be inserted at least partially into the cavity 3338. This configuration is shown in FIG. 8A which shows that the magnetic fastener component 3316, 3316B is at least partially positioned inside the cavity 3338 (for simplicities sake selected components of the fasteners halves 3312, 3314 are not shown in FIG. 8A).

FIGS. 8B to 8C show various fastener arrangements including a pairs of one of the first magnetic fastener components 3316, 3316B and one of the magnetic fastener components 3322. In these examples, the pairs of fastener halves 3312, 3314 are shown aligned with each other prior to the insertion portion 3316 being inserted into the cavity 3338.

FIG. 8D illustrates the magnetic fastener component 3316 of FIG. 6B engaged with the magnetic fastener component 3322 of FIG. 7C.

In embodiments, the magnetic fastener components 3316, 3322 are configured to allow the two components to be rotated relative to each other. This can facilitate adjustment of the orientation of the headgear strap 3304 and the patient interface 3050 relative to each other. This may facilitate better fit and comfort for a user. For instance, the magnetic fastener component 3316 is spherical and the magnetic fastener component 3316B is cylindrical, while the respective magnetic fastener component 3322 is sized and/or dimensioned to allow relative rotation.

It should be appreciated that in other forms of the present technology, the arrangement of the magnetic fastener components 3316, 3322 may be reversed, e.g. the magnetic fastener component 3322 is provided to the headgear strap 3304 and magnetic fastener component 3316 is provided to a support structure which is provided to the patient interface 3050.

### 4.3.3.3 Exposed Magnetic Fastener Component(s)

In embodiments, at least a portion of the magnetic fastener component(s) 3316, 3322 is exposed e.g. is not entirely covered by a layer of material.

For instance, in the embodiment illustrated in FIG. 9, a magnetic fastener component 3316 is attached to an outer surface of a headgear strap 3304. The magnetic fastener component 3316 may be welded, bonded, adhered, or otherwise attached to the outer surface of the headgear strap 3304.

### 4.3.3.4 Covered or Encapsulated Magnetic Fastener Component(s)

In embodiments, at least a portion of the magnetic fastener component(s) 3316, 3322 is/are covered by an additional the layer of material.

In the example shown in FIG. 10A, the additional layer of material 3328 is provided to and covers at least a portion of a surface of the first layer of material 3326. In these forms, the layers of material 3326, 3328 and magnetic fastener component 3316 form a part of the headgear strap 3304.

In other forms such as those shown in FIG. 10B, the magnetic fastener component 3322 may be at least partially covered, e.g. by a cap 3340. The cap 3340 may be a plastic material or a discrete piece of material which is fixed to a layer(s) forming the headgear strap or layer thereof to thereby encapsulate the magnetic fastener component 3322.

### 4.3.3.5 Magnetic Clips

In some forms, at least one of the first fastener half 3312 and second fastener half 3314 may be provided by a separate component, e.g. a separate magnetic clip structure that can be permanently or releasably attached to a headgear strap.

In embodiments, magnetic fasteners components 3316, 3322 may be provided in a separate magnetic clip such as first and second magnetic clips 3342, 3344 illustrated in FIGS. 9 and 10A. In these forms, the first magnetic clip 3342 may act as the first fastener half 3312, and the second magnetic clip 3344 may acts as the second fastener half 3314.

In these embodiments and the embodiments shown in FIGS. 11A to 11D, the magnetic clip 3342, 3344 includes a body 3346, 3348. The magnetic fastener component 3316, 3322 is provided to the body 3346, 3348 using any suitable technique. For instance, in one embodiment, the body 3346, 3348 may be a multilayer structure having a first layer of material and at least one other layer of material, with the magnetic fastener component 3316, 3322 encapsulated therebetween, as is discussed above with respect to FIGS. 6A to 6C.

Referring now to FIG. 11B which show views of a patient interface system 3000 including a positioning and stabilising structure 3300 having at least one magnetic clip 3342 according to the present technology.

In preferred embodiment, the magnetic clips 3342, 3344 are configured to attach e.g. releasably, to another component of the patient interface system 3000. For instance, the body 3346, 3348 may include a slot 3350, 3352. For instance, the slot 3350, 3352 can receive a headgear strap e.g. first lower headgear strap 3304 of a positioning and stabilising structure 3300, or be engaged by a corresponding hook/clip structure formed on one of a frame 3500 and a plenum chamber 3200.

In use, the magnetic fastener component 3316 engage(s) with a corresponding magnetic fastener component e.g. magnetic fastener component 3322 described herein with reference to FIG. 5A and FIG. 11B.

### 4.3.3.6 Protruding Magnetic Fastener Component(s)

It is also envisaged that in embodiments of the present technology, at least a portion of the magnetic fastener component 3316 may protrude beyond the additional layer of material 3328. For instance, a portion of the magnetic fastener component 3316 is exposed.

Referring to FIG. 9 which shows an embodiment in which a portion of the magnetic fastener component 3316 is exposed.

In examples, a magnetic fastener component 3316 is provided to the first layer of material 3326 such that at least a portion of the magnetic fastener component 3316 extends through a portion of the first layer of material 3326, e.g. a portion of the magnetic fastener component 3316 is embedded in the headgear strap 3304.

### 4.3.3.7 Multiple Magnetic Fastener Component(s)

In embodiments of the present technology, the first fastener half 3312 comprises at least one additional magnetic fastener component 3317. FIGS. 12A to 12C show part of a headgear strap 3304 which is provided with magnetic fastener components 3316, 3317.

In yet further embodiments of the present technology, the second fastener half 3314 comprises at least one additional magnetic fastener component 3323. FIG. 12D shows part of a support structures 3334 which is provided with magnetic fastener components 3322, 3323.

The provision of multiple magnetic fastener components may provide several advantages.

For instance, it may facilitate adjusting headgear strap length, e.g. the magnetic fastener component 3316 is detachable in use from engagement with a first magnetic fastener component 3322, and moveable in use to engage with a second magnetic fastener component 3323. In another embodiment, it may facilitate adjusting headgear strap orientation, e.g. a first magnetic fastener component 3316 is detachable in use from engagement with the magnetic fastener component 3322, and a second magnetic fastener component 3317 is moved in use into engagement with the magnetic fastener component 3322.

In addition, provision of multiple pairs of magnetic fastener components 3316, 3322 which concurrently engage each other may assist with limiting or preventing movement of two components relative to each other.

### 4.3.3.8 Alternative Magnetic Fastener Component(s)

In yet further embodiments of the present technology, the fastener arrangements may provide both a physical engagement and a magnetic engagement i.e. the fasteners halves 3312, 3314 provide some physical interaction which each other with resist disengagement between them in addition to the magnetic interaction of the magnetic fastener components 3316 and 3322. The physical interaction may be a press fit, snap-fit, friction fit, clipping structure or other physical fastener arrangement.

In an embodiment as illustrated in FIGS. 13A to 13C, one of the first and second fastener half 3312, 3314 may be configured with a male component 3354 and the other fastener half configured with a complementary female component 3356 which are configured to physically engage with each other. For instance, the male component 3354 may be a rivet structure provided to the headgear strap 3304 and the female component 3356 may be a rivet structure provided to the magnetic clip 3344. The male component 3354 comprises a press fit or snap fit component stud half provided to a headgear strap 3304 while the female component 3356 can be a complementary receiving structure 3358, e.g. a socket half provided in a magnetic clip 3344, on the frame 3500, or the plenum chamber 3200. In use, the male portion is inserted into the receiving structure and held in place by the press fit, snap-fit, friction fit, clipping structure or other physical fastener arrangement.

However, the male component 3354 and the female component 3356 may comprise other interlocking structure(s). In addition, the components may be reversed to have the male component on the magnetic clip 3344, on the frame 3500, or the plenum chamber 3200, while the female component 3356 may be provided on the headgear strap 3304.

In the embodiment shown in FIG. 13A, the male component 3354 is provided to the magnetic clip 3342 or headgear strap 3304 such that the magnet 3316 is held in place relative to the magnetic clip 3342 or headgear strap 3304. For example, the male component 3354 comprises a first part 3354a and an opposing second part 3354b which are configured to attach to opposing surfaces of the headgear strap 3304, and the magnet 3316 is held in place by the male component 3354. The headgear strap 3304 comprises an aperture 3360 through which the magnet 3316 extends. In the example shown, the first and second parts 3354a, 3354b are attached to the additional layer of material 3328, e.g. a layer of textile material 3328 and wherein the first layer of material 3326, e.g. a layer of foam material 3326 is provided thereto, as indicated by the arrow in FIG. 13A which is only for illustrative purposes.

In the embodiment shown in FIG. 13B, the female component 3356 is provided to the support structure 3334 or headgear strap 3304, and wherein the complementary receiving structure 3358 is configured to physically engage in use with the male component 3354. In the example shown, the female component 3356 comprises a first part 3356a which forms the receiving structure 3358 and an opposing second part 3356b configured to attach to the opposing surfaces of support structure 3334. In this example the support structure 3334 is formed of a first and additional layer of material, e.g. formed of a layer of foam material 3326 and a layer of textile material 3328.

FIG. 13C shows the position of the male component 3354 of FIG. 13A and the female component 3356 of FIG. 13B relative to each other when engaged. As illustrated, the magnetic fastener component 3316 is at least partially positioned in the cavity 3338. In these forms, the male component 3354 forms the insertion portion of the first fastener half 3312 which physically engages with the female component 3356 when inserted therein. It should therefore be appreciated that in some forms of the present technology the magnetic fastener component 3316 may not form the insertion portion of the first fastener half 3312.

### 4.3.4 Vent

In one form, the patient interface system 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO₂ by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

### 4.3.5 Decoupling Structure(s)

In one form the patient interface system 3000 includes at least one decoupling structure, for example, a swivel or a ball and socket.

### 4.3.6 Connection Port

Connection port 3600 allows for connection to the air circuit 4170.

### 4.3.7 Forehead Support

In one form, the patient interface system 3000 includes a forehead support 3700.

### 4.3.8 Anti-Asphyxia Valve

In one form, the patient interface system 3000 includes an anti-asphyxia valve.

### 4.3.9 Ports

In one form of the present technology, a patient interface system 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 4.3.10 Methods of Manufacture

The positioning and stabilising structure 3300 can be formed from a plurality of components which are attached together e.g. using sewing, bonding, adhesive, or ultrasonic welding to provide a desired configuration. Alternatively, the positioning and stabilising structure 3300 can be formed by cutting from a sheet of material a structure which provides the desired shape and configuration.

Referring now to FIG. 14 which shows representative steps in a method 6000 of manufacturing a strap for a positioning and stabilising structure 3300 having a fastener arrangement according to one form of the present technology.

The strap can be used as a first lower headgear strap 3304 or a second lower headgear strap 3306 of the positioning and stabilising structure 3300 of FIGS. 5A and 5B described herein. Alternatively, the strap can be for other parts of the positioning and stabilising structure 3300. For instance, the strap may be a crown strap 3302 of the positioning and stabilising structure 3300.

In general terms, the method 6000 includes the following steps in any order:
a. The step 6002 of selecting a first layer of material which will in use provide a patient contacting surface for the strap;
b. The step 6004 of positioning a magnetic fastener component and the first layer of material relative to each other; and
c. The step 6006 of attaching the magnetic fastener component to the first layer of material.

In addition, the method may optionally involve one or more of the following steps in any order:
d. The step of selecting at least one additional layer of material;
e. The step of selecting a further layer of material e.g. a layer of foam material;
f. The step of positioning the at least one additional layer of material and the further layer of material relative to the first layer of material;
g. The step of attaching at least one of the additional layer and the further layer of material to the first layer of material; and
h. The step of forming a desired shape for the strap.

In preferred forms, the step(s) of positioning the magnetic fastener component, the first layer of material and the additional layer of material positions the magnetic fastener component between the first layer and the additional layer.

It should be understood that one or more of the above steps may be performed completely or partially at the same time as each other. For instance, the step of adhering the first layer or material and the further layer of material may also form the desired shape for the strap e.g. as the layers of material are laminated together they are also cut to shape.

In addition, each step may be performed to produce multiple straps concurrently.

It is also envisaged that the method may involve the step of attaching the strap to at least one other component to form a portion of the positioning and stabilising structure 3300. For instance, this step may involve attaching the strap to a crown strap arrangement or other part of the positioning and stabilising structure 3300.

In embodiments of the technology, one or more of the layers may include an adhesive. For instance, one or more of the first layer of material and the additional layer of material may be coated with a heat sensitive adhesive. Alternatively, the foam layer may have a relatively low melting point. In these forms, during step (g), heat is applied to the layers of material to cause the adhesive or foam to at least partially melt to thereby adhere the layers together. This process secures the magnetic component to the layer(s) as the strap is formed.

Referring now to FIG. 15 which shows representative steps in a method 7000 of manufacturing at least a portion of a positioning and stabilising structure 3300 having a fastener component according to one form of the present technology.

The method 7000 may include one or more of the following steps in any order:
a. The step 7002 of providing at least one layer of material; and
b. The step 7004 of providing a magnetic fastener component to the at least one layer of material.

In preferred forms, the step 7002 includes providing a first layer of material and providing a second layer of material relative to the first layer of material. The first layer of material comprises a layer of foam, and/or the second layer is a layer of textile material, fabric or laminate material.

In some forms, the step 7004 includes positioning at least a portion of the fastener component between the first layer of material and the second layer of material.

In preferred forms, the step 7004 includes positioning the entire magnetic fastener component between the first layer of material and the second layer of material.

In addition, the method 7000 further includes the step of forming the first layer of material and the second layer of material together. In these forms, the step includes ultrasonic torsional welding, applying adhesive to one or more of the first and second layers of material, heat bonding and/or adhesive potting.

In preferred forms, the step 7004 includes attaching at least a portion of the magnetic fastener component to an outer surface of the at least one layer of material.

In preferred forms, the step 7004 includes forming a recess in a surface of the at least one layer of material. In these forms, the method further includes the step of positioning at least a portion of the magnetic fastener component in the recess formed in the at least one layer of material.

In some forms, the method further includes forming an aperture through the at least one layer of material. In these forms, the method may further include positioning at least a portion of the magnetic fastener component through the aperture formed in the at least one layer of material.

In preferred forms, step 7004 involves at least one of radio frequency welding, cutting, pressing or deforming the at least one layer of material to form the recess or aperture.

In preferred forms, the method further includes attaching the magnetic fastener component to the at least one layer of material such that the insertion portion of the fastener component protrudes away from the at least one layer of material.

In some forms, the method involves attaching the magnetic fastener component to the at least one layer of material such that a portion of the magnetic fastener component at least partially extends through the at least one layer of material.

In some forms, the method involves attaching the magnetic fastener component to the at least one layer of material such that a portion of the magnetic fastener component extends through the at least one layer of material from a first outer surface of the at least one layer of material to a second outer surface of the at least one layer of material.

In some forms, the methods involve providing at least two magnetic fastener components to the at least one layer of material. In these embodiments, the at least two magnetic fastener components may be provided to the same side of the at least one layer of material, or on different sides of the at least one layer of material.

In some forms, the magnetic fastener component comprises a stud half of a snap fastener or press stud which comprises a magnet, wherein the stud half forms the insertion portion. The stud half comprises a first part and an opposing second part configured to attach to opposing surfaces of the at least one layer of material. In these forms, the step 7004 includes:
a. attaching the first and second part of the stud half to opposing surfaces of the at least one layer of material such that the magnet is positioned between the first part and the opposing second part, wherein the step of attaching the first and second part of the stud half includes forming an aperture through the at least one layer of material;
b. positioning the magnet between the first part and opposing second part; and
c. attaching the first part to a first surface of the at least one layer of material and attaching the opposing second part to an opposing second surface of the at least one layer of material to secure the first and second part relative to each other which secures the magnet between the first and second part and relative to the at least one layer of material.

### 4.3.11 Conduit Headgear Patient Interface System

Referring now to FIG. 16 which illustrates an alternative embodiment of a patient interface system 3000A in accordance with one form of the present technology. In FIG. 16 like references to those used with reference to other Figures refer to like or similar components.

In the embodiment of FIG. 16, the patient interface system 3000A is in the form of a compact full-face patient interface 3050 having a seal forming structure 3100 configured to create a seal with or around a patient's nose and mouth. The seal-forming structure 3100 may have a nasal portion 3110 which seals with or around the patient's nose, and an oral portion (not shown) which seals with or around the patient's mouth. In some forms, the seal-forming structure 3100 includes at least one opening 3120. For example, the seal-forming structure 3100 may include a single opening and seals around the patient's nose and mouth. In some forms, the seal-forming structure 3100 may include a plurality of openings. For example, the nasal portion 3110 may comprise one opening or a pair of openings and seals around the patient's nares, and the oral portion may comprise a single opening and seals around the patient's mouth. In some forms, the seal-forming structure 3100 may have a nasal portion 3110 in the form of a nasal cradle. In some forms, the seal-forming structure 3100 may be substantially as described in International Application No. PCT/AU2019/050278, the entire contents of which are incorporated herein by reference.

As illustrated in the embodiment of FIG. 16, in some forms, the patient interface system 3000A may comprise a positioning and stabilising structure 3300 which has at least one headgear tube 3370, preferably a pair of headgear tubes 3370. The headgear tubes 3370 are configured to fluidly connect to the plenum chamber 3200 and supply the flow of pressurised breathable gas to the plenum chamber 3200. For example, the superior end regions 3372 of the headgear tubes 3370 may be connected to each other. The headgear tubes 3370 may include an inlet 3376 to which a connection port as described herein can be attached. Inferior end regions 3374 of each headgear tube 3370 may be connected to a respective inlet (not shown) formed on the plenum chamber 3200. In some forms, a connector 3378 may facilitate the attachment of each headgear tube 3370 to the plenum chamber 3200 as shown in FIG. 16.

In other forms, the headgear tubes 3370 may be releasably attached or permanently attached to the plenum chamber 3200. In yet other forms not shown, the headgear tubes 3370 may be integrally formed with the plenum chamber 3200, e.g. by co-moulding or moulding.

As illustrated in FIG. 16, in some forms, the headgear strap 3304 and the headgear strap 3306 may be attached to the patient interface 3050 as described above, e.g. they are provided with complementary fastener halves 3312, 3314. In some forms, headgear straps 3308, 3310 may be attached to the respective headgear tubes 3370, e.g. via a connector 3371 as illustrated in FIG. 16. In other forms (not shown), the headgear strap 3308 and the headgear strap 3310 may be attached to the respective headgear tubes 3370 using one of the fastener arrangements described herein.

### 4.4 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 6 cmH₂O, or at least 10cmH₂O, or at least 20 cmH₂O.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying air at positive pressure (e.g., a blower 4142), an outlet muffler 4124 and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of, the chassis 4016.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 4.4.1 RPT Device Mechanical & Pneumatic Components

An RPT device may comprise one or more of the following components in an integral unit. In an alternative form, one or more of the following components may be located as respective separate units.

### 4.4.1.1 Air Filter(s)

An RPT device in accordance with one form of the present technology may include an air filter 4110, or a plurality of air filters 4110.

In one form, an inlet air filter 4112 is located at the beginning of the pneumatic path upstream of a pressure generator 4140.

In one form, an outlet air filter 4114, for example an antibacterial filter, is located between an outlet of the pneumatic block 4020 and a patient interface system 3000 or 3800.

### 4.4.1.2 Muffler(s)

An RPT device in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface system 3000 or 3800.

### 4.4.1.3 Pressure Generator

In one form of the present technology, a pressure generator 4140 for producing a flow, or a supply, of air at positive pressure is a controllable blower 4142. For example the blower 4142 may include a brushless DC motor 4144 with one or more impellers. The impellers may be located in a volute. The blower may be capable of delivering a supply of air, for example at a rate of up to about 120 litres/minute, at a positive pressure in a range from about 4 cmH₂O to about 20 cmH₂O, or in other forms up to about 30 cmH₂O when delivering respiratory pressure therapy. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 4140 is under the control of the therapy device controller 4240.

In other forms, a pressure generator 4140 may be a piston-driven pump, a pressure regulator connected to a high pressure source (e.g. compressed air reservoir), or a bellows.

### 4.4.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 may be constructed and arranged to generate signals representing properties of the flow of air such as a flow rate, a pressure or a temperature at that point in the pneumatic path.

In one form of the present technology, one or more transducers 4270 may be located proximate to the patient interface system 3000 or 3800.

In one form, a signal from a transducer 4270 may be filtered, such as by low-pass, high-pass or band-pass filtering.

### 4.4.1.4.1 Flow Rate Sensor

A flow rate sensor 4274 in accordance with the present technology may be based on a differential pressure transducer, for example, an SDP600 Series differential pressure transducer from SENSIRION.

In one form, a signal generated by the flow rate sensor 4274 and representing a flow rate is received by the central controller 4230.

### 4.4.1.4.2 Pressure Sensor

A pressure sensor 4272 in accordance with the present technology is located in fluid communication with the pneumatic path. An example of a suitable pressure sensor is a transducer from the HONEYWELL ASDX series. An alternative suitable pressure sensor is a transducer from the NPA Series from GENERAL ELECTRIC.

In one form, a signal generated by the pressure sensor 4272 is received by the central controller 4230.

### 4.4.1.4.3 Motor Speed Transducer

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 4.4.1.5 Anti-Spill Back Valve

In one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 4.4.2 RPT Device Electrical Components

### 4.4.2.1 Power Supply

A power supply 4210 may be located internal or external of the external housing 4010 of the RPT device 4000.

In one form of the present technology, power supply 4210 provides electrical power to the RPT device 4000 only. In another form of the present technology, power supply 4210 provides electrical power to both RPT device 4000 and humidifier 5000.

### 4.4.2.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form, the input device 4220 may be constructed and arranged to allow a person to select a value and/or a menu option.

### 4.4.2.3 Central Controller

In one form of the present technology, the central controller 4230 is one or a plurality of processors suitable to control an RPT device 4000.

Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 4230 is a dedicated electronic circuit.

In one form, the central controller 4230 is an application-specific integrated circuit. In another form, the central controller 4230 comprises discrete electronic components.

The central controller 4230 may be configured to receive input signal(s) from one or more transducers 4270, one or more input devices 4220, and the humidifier 5000.

The central controller 4230 may be configured to provide output signal(s) to one or more of an output device 4290, a therapy device controller 4240, a data communication interface 4280, and the humidifier 5000.

In some forms of the present technology, the central controller 4230 is configured to implement the one or more methodologies described herein, such as the one or more algorithms 4300 expressed as computer programs stored in a non-transitory computer readable storage medium, such as memory 4260. In some forms of the present technology, the central controller 4230 may be integrated with an RPT device 4000. However, in some forms of the present technology, some methodologies may be performed by a remotely located device. For example, the remotely located device may determine control settings for a ventilator or detect respiratory related events by analysis of stored data such as from any of the sensors described herein.

### 4.4.2.4 Clock

The RPT device 4000 may include a clock 4232 that is connected to the central controller 4230.

### 4.4.2.5 Therapy Device Controller

In one form of the present technology, therapy device controller 4240 is a therapy control module 4330 that forms part of the algorithms 4300 executed by the central controller 4230.

In one form of the present technology, therapy device controller 4240 is a dedicated motor control integrated circuit. For example, in one form a MC33035 brushless DC motor controller, manufactured by ONSEMI is used.

### 4.4.2.6 Protection Circuits

The one or more protection circuits 4250 in accordance with the present technology may comprise an electrical protection circuit, a temperature and/or pressure safety circuit.

### 4.4.2.7 Memory

In accordance with one form of the present technology the RPT device 4000 includes memory 4260, e.g., non-volatile memory. In some forms, memory 4260 may include battery powered static RAM. In some forms, memory 4260 may include volatile RAM.

Memory 4260 may be located on the PCBA 4202. Memory 4260 may be in the form of EEPROM, or NAND flash.

Additionally or alternatively, RPT device 4000 includes a removable form of memory 4260, for example a memory card made in accordance with the Secure Digital (SD) standard.

In one form of the present technology, the memory 4260 acts as a non-transitory computer readable storage medium on which is stored computer program instructions expressing the one or more methodologies described herein, such as the one or more algorithms 4300.

### 4.4.2.8 Data Communication Systems

In one form of the present technology, a data communication interface 4280 is provided, and is connected to the central controller 4230. Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g. via Ethernet, or optical fibre) or a wireless protocol (e.g. CDMA, GSM, LTE) to connect to the Internet.

In one form, local external communication network 4284 utilises one or more communication standards, such as Bluetooth, or a consumer infrared protocol.

In one form, remote external device 4286 is one or more computers, for example a cluster of networked computers. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet or remote control.

### 4.4.2.9 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 4.4.2.9.1 Display Driver

A display driver 4292 receives as an input the characters, symbols, or images intended for display on the display 4294, and converts them to commands that cause the display 4294 to display those characters, symbols, or images.

### 4.4.2.9.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

### 4.4.3 RPT Device Algorithms

As mentioned above, in some forms of the present technology, the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and/or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 4.4.3.1 Pre-Processing Module

A pre-processing module 4310 in accordance with one form of the present technology receives as an input a signal from a transducer 4270, for example a flow rate sensor 4274 or pressure sensor 4272, and performs one or more process steps to calculate one or more output values that will be used as an input to another module, for example a therapy engine module 4320.

In one form of the present technology, the output values include the interface pressure Pm, the respiratory flow rate Qr, and the leak flow rate Ql.

In various forms of the present technology, the pre-processing module 4310 comprises one or more of the following algorithms: interface pressure estimation 4312, vent flow rate estimation 4314, leak flow rate estimation 4316, and respiratory flow rate estimation 4318.

### 4.4.3.1.1 Interface Pressure Estimation

In one form of the present technology, an interface pressure estimation algorithm 4312 receives as inputs a signal from the pressure sensor 4272 indicative of the pressure in the pneumatic path proximal to an outlet of the pneumatic block (the device pressure Pd) and a signal from the flow rate sensor 4274 representative of the flow rate of the airflow leaving the RPT device 4000 (the device flow rate Qd). The device flow rate Qd, absent any supplementary gas 4180, may be used as the total flow rate Qt. The interface pressure algorithm 4312 estimates the pressure drop ΔP through the air circuit 4170. The dependence of the pressure drop ΔP on the total flow rate Qt may be modelled for the particular air circuit 4170 by a pressure drop characteristic ΔP(Q). The interface pressure estimation algorithm, 4312 then provides as an output an estimated pressure, Pm, in the patient interface system 3000 or 3800. The pressure, Pm, in the patient interface system 3000 or 3800 may be estimated as the device pressure Pd minus the air circuit pressure drop ΔP.

### 4.4.3.1.2 Vent Flow Rate Estimation

In one form of the present technology, a vent flow rate estimation algorithm 4314 receives as an input an estimated pressure, Pm, in the patient interface system 3000 or 3800 from the interface pressure estimation algorithm 4312 and estimates a vent flow rate of air, Qv, from a vent 3400 in a patient interface system 3000 or 3800. The dependence of the vent flow rate Qv on the interface pressure Pm for the particular vent 3400 in use may be modelled by a vent characteristic Qv(Pm).

### 4.4.3.1.3 Leak Flow Rate Estimation

In one form of the present technology, a leak flow rate estimation algorithm 4316 receives as an input a total flow rate, Qt, and a vent flow rate Qv, and provides as an output an estimate of the leak flow rate Ql. In one form, the leak flow rate estimation algorithm estimates the leak flow rate Ql by calculating an average of the difference between total flow rate Qt and vent flow rate Qv over a period sufficiently long to include several breathing cycles, e.g. about 10 seconds.

In one form, the leak flow rate estimation algorithm 4316 receives as an input a total flow rate Qt, a vent flow rate Qv, and an estimated pressure, Pm, in the patient interface system 3000 or 3800, and provides as an output a leak flow rate Ql, by calculating a leak conductance, and determining a leak flow rate Ql to be a function of leak conductance and pressure, Pm. Leak conductance is calculated as the quotient of low pass filtered non-vent flow rate equal to the difference between total flow rate Qt and vent flow rate Qv, and low pass filtered square root of pressure Pm, where the low pass filter time constant has a value sufficiently long to include several breathing cycles, e.g. about 10 seconds. The leak flow rate Ql may be estimated as the product of leak conductance and a function of pressure, Pm.

### 4.4.3.1.4 Respiratory Flow Rate Estimation

In one form of the present technology, a respiratory flow rate estimation algorithm 4318 receives as an input a total flow rate, Qt, a vent flow rate, Qv, and a leak flow rate, Ql, and estimates a respiratory flow rate of air, Qr, to the patient, by subtracting the vent flow rate Qv and the leak flow rate Ql from the total flow rate Qt.

### 4.4.3.2 Therapy Engine Module

In one form of the present technology, a therapy engine module 4320 receives as inputs one or more of a pressure, Pm, in a patient interface system 3000 or 3800, and a respiratory flow rate of air to a patient, Qr, and provides as an output one or more therapy parameters.

In one form of the present technology, a therapy parameter is a treatment pressure Pt.

In one form of the present technology, therapy parameters are one or more of an amplitude of a pressure variation, a base pressure, and a target ventilation.

In various forms, the therapy engine module 4320 comprises one or more of the following algorithms: phase determination 4321, waveform determination 4322, ventilation determination 4323, inspiratory flow limitation determination 4324, apnea/hypopnea determination 4325, snore determination 4326, airway patency determination 4327, target ventilation determination 4328, and therapy parameter determination 4329.

### 4.4.3.2.1 Phase Determination

In one form of the present technology, the RPT device 4000 does not determine phase.

In one form of the present technology, a phase determination algorithm 4321 receives as an input a signal indicative of respiratory flow rate, Qr, and provides as an output a phase Φ of a current breathing cycle of a patient 1000.

In some forms, known as discrete phase determination, the phase output Φ is a discrete variable. One implementation of discrete phase determination provides a bi-valued phase output Φ with values of either inhalation or exhalation, for example represented as values of 0 and 0.5 revolutions respectively, upon detecting the start of spontaneous inhalation and exhalation respectively. RPT devices 4000 that “trigger” and “cycle” effectively perform discrete phase determination, since the trigger and cycle points are the instants at which the phase changes from exhalation to inhalation and from inhalation to exhalation, respectively. In one implementation of bi-valued phase determination, the phase output Φ is determined to have a discrete value of 0 (thereby “triggering” the RPT device 4000) when the respiratory flow rate Qr has a value that exceeds a positive threshold, and a discrete value of 0.5 revolutions (thereby “cycling” the RPT device 4000) when a respiratory flow rate Qr has a value that is more negative than a negative threshold. The inhalation time Ti and the exhalation time Te may be estimated as typical values over many respiratory cycles of the time spent with phase Φ equal to 0 (indicating inspiration) and 0.5 (indicating expiration) respectively.

Another implementation of discrete phase determination provides a tri-valued phase output Φ with a value of one of inhalation, mid-inspiratory pause, and exhalation.

In other forms, known as continuous phase determination, the phase output Φ is a continuous variable, for example varying from 0 to 1 revolutions, or 0 to 27c radians. RPT devices 4000 that perform continuous phase determination may trigger and cycle when the continuous phase reaches 0 and 0.5 revolutions, respectively. In one implementation of continuous phase determination, a continuous value of phase Φ is determined using a fuzzy logic analysis of the respiratory flow rate Qr. A continuous value of phase determined in this implementation is often referred to as “fuzzy phase”. In one implementation of a fuzzy phase determination algorithm 4321, the following rules are applied to the respiratory flow rate Qr:
1. If the respiratory flow rate is zero and increasing fast then the phase is 0 revolutions.
2. If the respiratory flow rate is large positive and steady then the phase is 0.25 revolutions.
3. If the respiratory flow rate is zero and falling fast, then the phase is 0.5 revolutions.
4. If the respiratory flow rate is large negative and steady then the phase is 0.75 revolutions.
5. If the respiratory flow rate is zero and steady and the 5-second low-pass filtered absolute value of the respiratory flow rate is large then the phase is 0.9 revolutions.
6. If the respiratory flow rate is positive and the phase is expiratory, then the phase is 0 revolutions.
7. If the respiratory flow rate is negative and the phase is inspiratory, then the phase is 0.5 revolutions.
8. If the 5-second low-pass filtered absolute value of the respiratory flow rate is large, the phase is increasing at a steady rate equal to the patient's breathing rate, low-pass filtered with a time constant of 20 seconds.

The output of each rule may be represented as a vector whose phase is the result of the rule and whose magnitude is the fuzzy extent to which the rule is true. The fuzzy extent to which the respiratory flow rate is “large”, “steady”, etc. is determined with suitable membership functions. The results of the rules, represented as vectors, are then combined by some function such as taking the centroid. In such a combination, the rules may be equally weighted, or differently weighted.

In another implementation of continuous phase determination, the phase Φ is first discretely estimated from the respiratory flow rate Qr as described above, as are the inhalation time Ti and the exhalation time Te. The continuous phase Φ at any instant may be determined as the half the proportion of the inhalation time Ti that has elapsed since the previous trigger instant, or 0.5 revolutions plus half the proportion of the exhalation time Te that has elapsed since the previous cycle instant (whichever instant was more recent).

### 4.4.3.2.2 Waveform Determination

In one form of the present technology, the therapy parameter determination algorithm 4329 provides an approximately constant treatment pressure throughout a respiratory cycle of a patient.

In other forms of the present technology, the therapy control module 4330 controls the pressure generator 4140 to provide a treatment pressure Pt that varies as a function of phase Φ of a respiratory cycle of a patient according to a waveform template Π(Φ).

In one form of the present technology, a waveform determination algorithm 4322 provides a waveform template Π(Φ) with values in the range [0, 1] on the domain of phase values Φ provided by the phase determination algorithm 4321 to be used by the therapy parameter determination algorithm 4329.

In one form, suitable for either discrete or continuously-valued phase, the waveform template Π(Φ) is a square-wave template, having a value of 1 for values of phase up to and including 0.5 revolutions, and a value of 0 for values of phase above 0.5 revolutions. In one form, suitable for continuously-valued phase, the waveform template Π(Φ) comprises two smoothly curved portions, namely a smoothly curved (e.g. raised cosine) rise from 0 to 1 for values of phase up to 0.5 revolutions, and a smoothly curved (e.g. exponential) decay from 1 to 0 for values of phase above 0.5 revolutions. In one form, suitable for continuously-valued phase, the waveform template Π(Φ) is based on a square wave, but with a smooth rise from 0 to 1 for values of phase up to a “rise time” that is less than 0.5 revolutions, and a smooth fall from 1 to 0 for values of phase within a “fall time” after 0.5 revolutions, with a “fall time” that is less than 0.5 revolutions.

In some forms of the present technology, the waveform determination algorithm 4322 selects a waveform template Π(Φ) from a library of waveform templates, dependent on a setting of the RPT device. Each waveform template Π(Φ) in the library may be provided as a lookup table of values II against phase values Φ. In other forms, the waveform determination algorithm 4322 computes a waveform template Π(Φ) “on the fly” using a predetermined functional form, possibly parametrised by one or more parameters (e.g. time constant of an exponentially curved portion). The parameters of the functional form may be predetermined or dependent on a current state of the patient 1000.

In some forms of the present technology, suitable for discrete bi-valued phase of either inhalation (Φ=0 revolutions) or exhalation (Φ=0.5 revolutions), the waveform determination algorithm 4322 computes a waveform template Π “on the fly” as a function of both discrete phase Φ and time t measured since the most recent trigger instant. In one such form, the waveform determination algorithm 4322 computes the waveform template Π(Φ, t) in two portions (inspiratory and expiratory) as follows:

$$Π ( Φ , t ) = \{ \begin{matrix}{Π}_{i} ( t ) , & Φ = 0 \\ {Π}_{e} ( t - {T}_{i} ) , & Φ = 0.5\end{matrix}$$

where Πᵢ(t) and Πₑ(t) are inspiratory and expiratory portions of the waveform template Π(Φ, t). In one such form, the inspiratory portion Hᵢ(t) of the waveform template is a smooth rise from 0 to 1 parametrised by a rise time, and the expiratory portion Πₑ(t) of the waveform template is a smooth fall from 1 to 0 parametrised by a fall time.

### 4.4.3.2.3 Ventilation Determination

In one form of the present technology, a ventilation determination algorithm 4323 receives an input a respiratory flow rate Qr, and determines a measure indicative of current patient ventilation, Vent.

In some implementations, the ventilation determination algorithm 4323 determines a measure of ventilation Vent that is an estimate of actual patient ventilation. One such implementation is to take half the absolute value of respiratory flow rate, Qr, optionally filtered by low-pass filter such as a second order Bessel low-pass filter with a corner frequency of 0.11 Hz.

In other implementations, the ventilation determination algorithm 4323 determines a measure of ventilation Vent that is broadly proportional to actual patient ventilation. One such implementation estimates peak respiratory flow rate Qpeak over the inspiratory portion of the cycle. This and many other procedures involving sampling the respiratory flow rate Qr produce measures which are broadly proportional to ventilation, provided the flow rate waveform shape does not vary very much (here, the shape of two breaths is taken to be similar when the flow rate waveforms of the breaths normalised in time and amplitude are similar). Some simple examples include the median positive respiratory flow rate, the median of the absolute value of respiratory flow rate, and the standard deviation of flow rate. Arbitrary linear combinations of arbitrary order statistics of the absolute value of respiratory flow rate using positive coefficients, and even some using both positive and negative coefficients, are approximately proportional to ventilation. Another example is the mean of the respiratory flow rate in the middle K proportion (by time) of the inspiratory portion, where 0<K<1. There is an arbitrarily large number of measures that are exactly proportional to ventilation if the flow rate shape is constant.

### 4.4.3.2.4 Determination of Inspiratory Flow Limitation

In one form of the present technology, the central controller 4230 executes an inspiratory flow limitation determination algorithm 4324 for the determination of the extent of inspiratory flow limitation.

In one form, the inspiratory flow limitation determination algorithm 4324 receives as an input a respiratory flow rate signal Qr and provides as an output a metric of the extent to which the inspiratory portion of the breath exhibits inspiratory flow limitation.

In one form of the present technology, the inspiratory portion of each breath is identified by a zero-crossing detector. A number of evenly spaced points (for example, sixty-five), representing points in time, are interpolated by an interpolator along the inspiratory flow rate-time curve for each breath. The curve described by the points is then scaled by a scalar to have unity length (duration/period) and unity area to remove the effects of changing breathing rate and depth. The scaled breaths are then compared in a comparator with a pre-stored template representing a normal unobstructed breath, similar to the inspiratory portion of the breath shown in FIG. 6A. Breaths deviating by more than a specified threshold (typically 1 scaled unit) at any time during the inspiration from this template, such as those due to coughs, sighs, swallows and hiccups, as determined by a test element, are rejected. For non-rejected data, a moving average of the first such scaled point is calculated by the central controller 4230 for the preceding several inspiratory events. This is repeated over the same inspiratory events for the second such point, and so on. Thus, for example, sixty five scaled data points are generated by the central controller 4230, and represent a moving average of the preceding several inspiratory events, e.g., three events. The moving average of continuously updated values of the (e.g., sixty five) points are hereinafter called the “scaled flow rate”, designated as Qs(t). Alternatively, a single inspiratory event can be utilised rather than a moving average.

From the scaled flow rate, two shape factors relating to the determination of partial obstruction may be calculated.

Shape factor 1 is the ratio of the mean of the middle (e.g. thirty-two) scaled flow rate points to the mean overall (e.g. sixty-five) scaled flow rate points. Where this ratio is in excess of unity, the breath will be taken to be normal. Where the ratio is unity or less, the breath will be taken to be obstructed. A ratio of about 1.17 is taken as a threshold between partially obstructed and unobstructed breathing, and equates to a degree of obstruction that would permit maintenance of adequate oxygenation in a typical patient.

Shape factor 2 is calculated as the RMS deviation from unit scaled flow rate, taken over the middle (e.g. thirty two) points. An RMS deviation of about 0.2 units is taken to be normal. An RMS deviation of zero is taken to be a totally flow—limited breath. The closer the RMS deviation to zero, the breath will be taken to be more flow limited.

Shape factors 1 and 2 may be used as alternatives, or in combination. In other forms of the present technology, the number of sampled points, breaths and middle points may differ from those described above. Furthermore, the threshold values can be other than those described.

### 4.4.3.2.5 Determination of Apneas and Hypopneas

In one form of the present technology, the central controller 4230 executes an apnea/hypopnea determination algorithm 4325 for the determination of the presence of apneas and/or hypopneas.

In one form, the apnea/hypopnea determination algorithm 4325 receives as an input a respiratory flow rate signal Qr and provides as an output a flag that indicates that an apnea or a hypopnea has been detected.

In one form, an apnea will be said to have been detected when a function of respiratory flow rate Qr falls below a flow rate threshold for a predetermined period of time. The function may determine a peak flow rate, a relatively short-term mean flow rate, or a flow rate intermediate of relatively short-term mean and peak flow rate, for example an RMS flow rate. The flow rate threshold may be a relatively long-term measure of flow rate.

In one form, a hypopnea will be said to have been detected when a function of respiratory flow rate Qr falls below a second flow rate threshold for a predetermined period of time. The function may determine a peak flow, a relatively short-term mean flow rate, or a flow rate intermediate of relatively short-term mean and peak flow rate, for example an RMS flow rate. The second flow rate threshold may be a relatively long-term measure of flow rate. The second flow rate threshold is greater than the flow rate threshold used to detect apneas.

### 4.4.3.2.6 Determination of Snore

In one form of the present technology, the central controller 4230 executes one or more snore determination algorithms 4326 for the determination of the extent of snore.

In one form, the snore determination algorithm 4326 receives as an input a respiratory flow rate signal Qr and provides as an output a metric of the extent to which snoring is present.

The snore determination algorithm 4326 may comprise the step of determining the intensity of the flow rate signal in the range of 30-300 Hz. Further, the snore determination algorithm 4326 may comprise a step of filtering the respiratory flow rate signal Qr to reduce background noise, e.g., the sound of airflow in the system from the blower.

### 4.4.3.2.7 Determination of Airway Patency

In one form of the present technology, the central controller 4230 executes one or more airway patency determination algorithms 4327 for the determination of the extent of airway patency.

In one form, the airway patency determination algorithm 4327 receives as an input a respiratory flow rate signal Qr, and determines the power of the signal in the frequency range of about 0.75 Hz and about 3 Hz. The presence of a peak in this frequency range is taken to indicate an open airway. The absence of a peak is taken to be an indication of a closed airway.

In one form, the frequency range within which the peak is sought is the frequency of a small forced oscillation in the treatment pressure Pt. In one implementation, the forced oscillation is of frequency 2 Hz with amplitude about 1 cmH₂O.

In one form, airway patency determination algorithm 4327 receives as an input a respiratory flow rate signal Qr, and determines the presence or absence of a cardiogenic signal. The absence of a cardiogenic signal is taken to be an indication of a closed airway.

### 4.4.3.2.8 Determination of Target Ventilation

In one form of the present technology, the central controller 4230 takes as input the measure of current ventilation, Vent, and executes one or more target ventilation determination algorithms 4328 for the determination of a target value Vtgt for the measure of ventilation.

In some forms of the present technology, there is no target ventilation determination algorithm 4328, and the target value Vtgt is predetermined, for example by hard-coding during configuration of the RPT device 4000 or by manual entry through the input device 4220.

In other forms of the present technology, such as adaptive servo-ventilation (ASV), the target ventilation determination algorithm 4328 computes a target value Vtgt from a value Vtyp indicative of the typical recent ventilation of the patient.

In some forms of adaptive servo-ventilation, the target ventilation Vtgt is computed as a high proportion of, but less than, the typical recent ventilation Vtyp. The high proportion in such forms may be in the range (80%, 100%), or (85%, 95%), or (87%, 92%).

In other forms of adaptive servo-ventilation, the target ventilation Vtgt is computed as a slightly greater than unity multiple of the typical recent ventilation Vtyp.

The typical recent ventilation Vtyp is the value around which the distribution of the measure of current ventilation Vent over multiple time instants over some predetermined timescale tends to cluster, that is, a measure of the central tendency of the measure of current ventilation over recent history. In one implementation of the target ventilation determination algorithm 4328, the recent history is of the order of several minutes, but in any case should be longer than the timescale of Cheyne-Stokes waxing and waning cycles. The target ventilation determination algorithm 4328 may use any of the variety of well-known measures of central tendency to determine the typical recent ventilation Vtyp from the measure of current ventilation, Vent. One such measure is the output of a low-pass filter on the measure of current ventilation Vent, with time constant equal to one hundred seconds.

### 4.4.3.2.9 Determination of Therapy Parameters

In some forms of the present technology, the central controller 4230 executes one or more therapy parameter determination algorithms 4329 for the determination of one or more therapy parameters using the values returned by one or more of the other algorithms in the therapy engine module 4320.

In one form of the present technology, the therapy parameter is an instantaneous treatment pressure Pt. In one implementation of this form, the therapy parameter determination algorithm 4329 determines the treatment pressure Pt using the equation

Pt=AΠ(Φ,t)+P₀ (1)

where:
A is the amplitude,
Π(Φ, t) is the waveform template value (in the range 0 to 1) at the current value Φ of phase and t of time, and
P₀ is a base pressure.

If the waveform determination algorithm 4322 provides the waveform template Π(Φ, t) as a lookup table of values Π indexed by phase Φ, the therapy parameter determination algorithm 4329 applies equation (1) by locating the nearest lookup table entry to the current value Φ of phase returned by the phase determination algorithm 4321, or by interpolation between the two entries straddling the current value Φ of phase.

The values of the amplitude A and the base pressure P₀ may be set by the therapy parameter determination algorithm 4329 depending on the chosen respiratory pressure therapy mode in the manner described below.

### 4.4.3.3 Therapy Control Module

The therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of air whose interface pressure Pm at the patient interface system 3000 or 3800 is equal to the treatment pressure Pt.

### 4.4.3.4 Detection of Fault Conditions

In one form of the present technology, the central controller 4230 executes one or more methods 4340 for the detection of fault conditions. The fault conditions detected by the one or more methods 4340 may include at least one of the following:
Power failure (no power, or insufficient power)
Transducer fault detection
Failure to detect the presence of a component
Operating parameters outside recommended ranges (e.g. pressure, flow rate, temperature, PaO₂)
Failure of a test alarm to generate a detectable alarm signal.

In an example, the failure to detect the presence of a component may include failure to detect engagement between the fastener components according to any of the forms of the present technology.

Upon detection of the fault condition, the corresponding algorithm 4340 signals the presence of the fault by one or more of the following:
Initiation of an audible, visual &/or kinetic (e.g. vibrating) alarm
Sending a message to an external device
Logging of the incident

### 4.5 Air Circuit

An air circuit 4170 in accordance with an aspect of the present technology is a conduit or a tube constructed and arranged to allow, in use, a flow of air to travel between two components such as RPT device 4000 and the patient interface system 3000 or 3800.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller 4230. One example of an air circuit 4170 comprising a heated wire circuit is described in U.S. Pat. No. 8,733,349, which is incorporated herewithin in its entirety by reference.

### 4.6 Respiratory Therapy Modes

Various respiratory therapy modes may be implemented by the disclosed respiratory therapy system.

### 4.6.1 CPAP Therapy

In some implementations of respiratory pressure therapy, the central controller 4230 sets the treatment pressure Pt according to the treatment pressure equation (1) as part of the therapy parameter determination algorithm 4329. In one such implementation, the amplitude A is identically zero, so the treatment pressure Pt (which represents a target value to be achieved by the interface pressure Pm at the current instant of time) is identically equal to the base pressure P₀ throughout the respiratory cycle. Such implementations are generally grouped under the heading of CPAP therapy. In such implementations, there is no need for the therapy engine module 4320 to determine phase Φ or the waveform template Π(Φ).

In CPAP therapy, the base pressure P₀ may be a constant value that is hard-coded or manually entered to the RPT device 4000. Alternatively, the central controller 4230 may repeatedly compute the base pressure P₀ as a function of indices or measures of sleep disordered breathing returned by the respective algorithms in the therapy engine module 4320, such as one or more of flow limitation, apnea, hypopnea, patency, and snore. This alternative is sometimes referred to as APAP therapy.

FIG. 4E is a flow chart illustrating a method 4500 carried out by the central controller 4230 to continuously compute the base pressure P₀ as part of an APAP therapy implementation of the therapy parameter determination algorithm 4329, when the pressure support A is identically zero.

The method 4500 starts at step 4520, at which the central controller 4230 compares the measure of the presence of apnea/hypopnea with a first threshold, and determines whether the measure of the presence of apnea/hypopnea has exceeded the first threshold for a predetermined period of time, indicating an apnea/hypopnea is occurring. If so, the method 4500 proceeds to step 4540; otherwise, the method 4500 proceeds to step 4530. At step 4540, the central controller 4230 compares the measure of airway patency with a second threshold. If the measure of airway patency exceeds the second threshold, indicating the airway is patent, the detected apnea/hypopnea is deemed central, and the method 4500 proceeds to step 4560; otherwise, the apnea/hypopnea is deemed obstructive, and the method 4500 proceeds to step 4550.

At step 4530, the central controller 4230 compares the measure of flow limitation with a third threshold. If the measure of flow limitation exceeds the third threshold, indicating inspiratory flow is limited, the method 4500 proceeds to step 4550; otherwise, the method 4500 proceeds to step 4560.

At step 4550, the central controller 4230 increases the base pressure P₀ by a predetermined pressure increment ΔP, provided the resulting treatment pressure Pt would not exceed a maximum treatment pressure P max. In one implementation, the predetermined pressure increment ΔP and maximum treatment pressure P max are 1 cmH₂O and 25 cmH₂O respectively. In other implementations, the pressure increment ΔP can be as low as 0.1 cmH₂O and as high as 3 cmH₂O, or as low as 0.5 cmH₂O and as high as 2 cmH₂O. In other implementations, the maximum treatment pressure P max can be as low as 15 cmH₂O and as high as 35 cmH₂O, or as low as 20 cmH₂O and as high as 30 cmH₂O. The method 4500 then returns to step 4520.

At step 4560, the central controller 4230 decreases the base pressure P₀ by a decrement, provided the decreased base pressure P₀ would not fall below a minimum treatment pressure P min. The method 4500 then returns to step 4520. In one implementation, the decrement is proportional to the value of P₀-P min, so that the decrease in P₀ to the minimum treatment pressure P min in the absence of any detected events is exponential. In one implementation, the constant of proportionality is set such that the time constant τ of the exponential decrease of P₀ is 60 minutes, and the minimum treatment pressure P min is 4 cmH₂O. In other implementations, the time constant τ could be as low as 1 minute and as high as 300 minutes, or as low as 5 minutes and as high as 180 minutes. In other implementations, the minimum treatment pressure P min can be as low as 0 cmH₂O and as high as 8 cmH₂O, or as low as 2 cmH₂O and as high as 6 cmH₂O. Alternatively, the decrement in P₀ could be predetermined, so the decrease in P₀ to the minimum treatment pressure P min in the absence of any detected events is linear.

### 4.6.2 Bi-Level Therapy

In other implementations of this form of the present technology, the value of amplitude A in equation (1) may be positive. Such implementations are known as bi-level therapy, because in determining the treatment pressure Pt using equation (1) with positive amplitude A, the therapy parameter determination algorithm 4329 oscillates the treatment pressure Pt between two values or levels in synchrony with the spontaneous respiratory effort of the patient 1000. That is, based on the typical waveform templates Π(Φ, t) described above, the therapy parameter determination algorithm 4329 increases the treatment pressure Pt to P₀+A (known as the IPAP) at the start of, or during, or inspiration and decreases the treatment pressure Pt to the base pressure P₀ (known as the EPAP) at the start of, or during, expiration.

In some forms of bi-level therapy, the IPAP is a treatment pressure that has the same purpose as the treatment pressure in CPAP therapy modes, and the EPAP is the IPAP minus the amplitude A, which has a “small” value (a few cmH₂O) sometimes referred to as the Expiratory Pressure Relief (EPR). Such forms are sometimes referred to as CPAP therapy with EPR, which is generally thought to be more comfortable than straight CPAP therapy. In CPAP therapy with EPR, either or both of the IPAP and the EPAP may be constant values that are hard-coded or manually entered to the RPT device 4000. Alternatively, the therapy parameter determination algorithm 4329 may repeatedly compute the IPAP and/or the EPAP during CPAP with EPR. In this alternative, the therapy parameter determination algorithm 4329 repeatedly computes the EPAP and/or the IPAP as a function of indices or measures of sleep disordered breathing returned by the respective algorithms in the therapy engine module 4320 in analogous fashion to the computation of the base pressure P₀ in APAP therapy described above.

In other forms of bi-level therapy, the amplitude A is large enough that the RPT device 4000 does some or all of the work of breathing of the patient 1000. In such forms, known as pressure support ventilation therapy, the amplitude A is referred to as the pressure support, or swing. In pressure support ventilation therapy, the IPAP is the base pressure P₀ plus the pressure support A, and the EPAP is the base pressure P₀.

In some forms of pressure support ventilation therapy, known as fixed pressure support ventilation therapy, the pressure support A is fixed at a predetermined value, e.g. 10 cmH₂O. The predetermined pressure support value is a setting of the RPT device 4000, and may be set for example by hard-coding during configuration of the RPT device 4000 or by manual entry through the input device 4220.

In other forms of pressure support ventilation therapy, broadly known as servo-ventilation, the therapy parameter determination algorithm 4329 takes as input some currently measured or estimated parameter of the respiratory cycle (e.g. the current measure Vent of ventilation) and a target value of that respiratory parameter (e.g. a target value Vtgt of ventilation) and repeatedly adjusts the parameters of equation (1) to bring the current measure of the respiratory parameter towards the target value. In a form of servo-ventilation known as adaptive servo-ventilation (ASV), which has been used to treat CSR, the respiratory parameter is ventilation, and the target ventilation value Vtgt is computed by the target ventilation determination algorithm 4328 from the typical recent ventilation Vtyp, as described above.

In some forms of servo-ventilation, the therapy parameter determination algorithm 4329 applies a control methodology to repeatedly compute the pressure support A so as to bring the current measure of the respiratory parameter towards the target value. One such control methodology is Proportional-Integral (PI) control. In one implementation of PI control, suitable for ASV modes in which a target ventilation Vtgt is set to slightly less than the typical recent ventilation Vtyp, the pressure support A is repeatedly computed as:

A=G∫(Vent−Vtgt)dt (2)

where G is the gain of the PI control. Larger values of gain G can result in positive feedback in the therapy engine module 4320. Smaller values of gain G may permit some residual untreated CSR or central sleep apnea. In some implementations, the gain G is fixed at a predetermined value, such as −0.4 cmH₂O/(L/min)/sec. Alternatively, the gain G may be varied between therapy sessions, starting small and increasing from session to session until a value that substantially eliminates CSR is reached. Conventional means for retrospectively analysing the parameters of a therapy session to assess the severity of CSR during the therapy session may be employed in such implementations In yet other implementations, the gain G may vary depending on the difference between the current measure Vent of ventilation and the target ventilation Vtgt.

Other servo-ventilation control methodologies that may be applied by the therapy parameter determination algorithm 4329 include proportional (P), proportional-differential (PD), and proportional-integral-differential (PID).

The value of the pressure support A computed via equation (2) may be clipped to a range defined as [Amin, Amax]. In this implementation, the pressure support A sits by default at the minimum pressure support Amin until the measure of current ventilation Vent falls below the target ventilation Vtgt, at which point A starts increasing, only falling back to Amin when Vent exceeds Vtgt once again.

The pressure support limits Amin and Amax are settings of the RPT device 4000, set for example by hard-coding during configuration of the RPT device 4000 or by manual entry through the input device 4220.

In pressure support ventilation therapy modes, the EPAP is the base pressure P₀. As with the base pressure P₀ in CPAP therapy, the EPAP may be a constant value that is prescribed or determined during titration. Such a constant EPAP may be set for example by hard-coding during configuration of the RPT device 4000 or by manual entry through the input device 4220. This alternative is sometimes referred to as fixed-EPAP pressure support ventilation therapy. Titration of the EPAP for a given patient may be performed by a clinician during a titration session with the aid of PSG, with the aim of preventing obstructive apneas, thereby maintaining an open airway for the pressure support ventilation therapy, in similar fashion to titration of the base pressure P₀ in constant CPAP therapy.

Alternatively, the therapy parameter determination algorithm 4329 may repeatedly compute the base pressure P₀ during pressure support ventilation therapy. In such implementations, the therapy parameter determination algorithm 4329 repeatedly computes the EPAP as a function of indices or measures of sleep disordered breathing returned by the respective algorithms in the therapy engine module 4320, such as one or more of flow limitation, apnea, hypopnea, patency, and snore. Because the continuous computation of the EPAP resembles the manual adjustment of the EPAP by a clinician during titration of the EPAP, this process is also sometimes referred to as auto-titration of the EPAP, and the therapy mode is known as auto-titrating EPAP pressure support ventilation therapy, or auto-EPAP pressure support ventilation therapy.

### 4.6.3 High Flow Therapy

In other forms of respiratory therapy, the pressure of the flow of air is not controlled as it is for respiratory pressure therapy. Rather, the central controller 4230 controls the pressure generator 4140 to deliver a flow of air whose device flow rate Qd is controlled to a treatment or target flow rate Qtgt that is typically positive throughout the patient's breathing cycle. Such forms are generally grouped under the heading of flow therapy. In flow therapy, the treatment flow rate Qtgt may be a constant value that is hard-coded or manually entered to the RPT device 4000. If the treatment flow rate Qtgt is sufficient to exceed the patient's peak inspiratory flow rate, the therapy is generally referred to as high flow therapy (HFT). Alternatively, the treatment flow rate may be a profile Qtgt(t) that varies over the respiratory cycle.

### 4.7 Glossary

For the purposes of the present technology disclosure, in certain forms of the present technology, one or more of the following definitions may apply. In other forms of the present technology, alternative definitions may apply.

### 4.7.1 General

Air: In certain forms of the present technology, air may be taken to mean atmospheric air, and in other forms of the present technology air may be taken to mean some other combination of breathable gases, e.g. atmospheric air enriched with oxygen.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar˜0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy (RPT): The application of a supply of air to an entrance to the airways at a treatment pressure that is typically positive with respect to atmosphere.

Ventilator: A mechanical device that provides pressure support to a patient to perform some or all of the work of breathing.

### 4.7.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240. (Year? Required?)

Polycarbonate: a thermoplastic polymer of Bisphenol-A Carbonate.

### 4.7.1.2 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH₂O pressure.

As an example, an I-beam may comprise a different bending stiffness (resistance to a bending load) in a first direction in comparison to a second, orthogonal direction. In another example, a structure or component may be floppy in a first direction and rigid in a second direction.

### 4.7.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:
(i) Flattened: Having a rise followed by a relatively flat portion, followed by a fall.
(ii) M-shaped: Having two local peaks, one at the leading edge, and one at the trailing edge, and a relatively flat portion between the two peaks.
(iii) Chair-shaped: Having a single local peak, the peak being at the leading edge, followed by a relatively flat portion.
(iv) Reverse-chair shaped: Having a relatively flat portion followed by single local peak, the peak being at the trailing edge.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Positive End-Expiratory Pressure (PEEP): The pressure above atmosphere in the lungs that exists at the end of expiration.

Peak flow rate (Qpeak): The maximum value of flow rate during the inspiratory portion of the respiratory flow waveform.

Respiratory flow rate, patient airflow rate, respiratory airflow rate (Qr): These terms may be understood to refer to the RPT device's estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

(inhalation) Time (Ti): The duration of the inspiratory portion of the respiratory flow rate waveform.

(exhalation) Time (Te): The duration of the expiratory portion of the respiratory flow rate waveform.

(total) Time (Ttot): The total duration between the start of one inspiratory portion of a respiratory flow rate waveform and the start of the following inspiratory portion of the respiratory flow rate waveform.

Typical recent ventilation: The value of ventilation around which recent values of ventilation Vent over some predetermined timescale tend to cluster, that is, a measure of the central tendency of the recent values of ventilation.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 4.7.3 Ventilation

Adaptive Servo-Ventilator (ASV): A servo-ventilator that has a changeable, rather than fixed target ventilation. The changeable target ventilation may be learned from some characteristic of the patient, for example, a respiratory characteristic of the patient.

Backup rate: A parameter of a ventilator that establishes the minimum breathing rate (typically in number of breaths per minute) that the ventilator will deliver to the patient, if not triggered by spontaneous respiratory effort.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired interface pressure which the ventilator will attempt to achieve at a given time.

End expiratory pressure (EEP): Desired interface pressure which the ventilator will attempt to achieve at the end of the expiratory portion of the breath. If the pressure waveform template Π(Φ) is zero-valued at the end of expiration, i.e. Π(Φ)=0 when Φ=1, the EEP is equal to the EPAP.

Inspiratory positive airway pressure (IPAP): Maximum desired interface pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP−EPAP). In some contexts pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Spontaneous/Timed (S/T): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator delivers a breath of air to a spontaneously breathing patient, it is said to be triggered to do so at the initiation of the respiratory portion of the breathing cycle by the patient's efforts.

### 4.7.4 Anatomy

### 4.7.4.1 Anatomy of the Face

Ala: the external outer wall or “wing” of each nostril (plural: alar)

Alar angle:

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Lip, lower (labrale inferius):

Lip, upper (labrale superius):

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

### 4.7.4.2 Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 4.7.4.3 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 4.7.5 Patient Interface

Anti-asphyxia valve (AAV): The component or sub-assembly of a mask system that, by opening to atmosphere in a failsafe manner, reduces the risk of excessive CO₂ rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Functional dead space: (description to be inserted here)

Headgear: Headgear will be taken to mean a form of positioning and stabilising structure designed for use on a head. For example the headgear may comprise a collection of one or more struts, ties and stiffeners configured to locate and retain a patient interface in position on a patient's face for delivery of respiratory therapy. Some ties are formed of a soft, flexible, elastic material such as a laminated composite of foam and fabric.

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 4.7.6 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

### 4.7.6.1 Holes

A surface may have a one-dimensional hole, e.g. a hole bounded by a plane curve or by a space curve. Thin structures (e.g. a membrane) with a hole, may be described as having a one-dimensional hole. See for example the one dimensional hole in the surface of structure shown in FIG. 31, bounded by a plane curve.

A structure may have a two-dimensional hole, e.g. a hole bounded by a surface. For example, an inflatable tyre has a two dimensional hole bounded by the interior surface of the tyre. In a yet another example, a conduit may comprise a one-dimension hole (e.g. at its entrance or at its exit), and a two-dimension hole bounded by the inside surface of the conduit. See also the two dimensional hole through the structure shown in FIG. 3K, bounded by a surface as shown.

### 4.8 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1. A positioning and stabilising structure for a patient interface system, comprising
a rear strap assembly,
at least one strap which extends away from the rear strap assembly and along a side of the patient's face, wherein the at least one strap has a distal end, and
a first strap fastener that comprises a magnetic fastener component provided to the at least one strap and that is positioned between the distal end and the rear strap assembly.

2. A positioning and stabilising structure for a patient interface system, comprising at least one strap and a strap fastener half, wherein the strap fastener half comprises a magnetic fastener component which is formed to the at least one strap.

3. A positioning and stabilising structure for a patient interface system, comprising at least one strap and a strap fastener half which is permanently attached to the positioning and stabilising structure, wherein the strap fastener half comprises a magnetic fastener component, and further wherein the strap fastener half is configured to in use engage with a corresponding fastener half on a patient interface to attach the positioning and stabilising structure to the patient interface.

4. The positioning and stabilising structure as claimed in claim 1, comprising a first strap and a second strap, wherein the first strap comprises a first strap fastener half and the second strap comprises a second strap fastener half, and further wherein the first strap fastener half and the second strap fastener half each comprises a magnetic fastener component.

5. The positioning and stabilising structure as claimed in claim 4, wherein the magnetic fastener components are both permanently attached to, or formed to, the respective strap.

6. The positioning and stabilising structure as claimed in claim 4, wherein at least one of the first strap fastener half and the second strap fastener half comprises an insertion portion configured to in use be inserted into a corresponding receiving portion of a patient interface fastener half.

7. The positioning and stabilising structure as claimed in claim 1, wherein the strap fastener half is attached to a first layer of material.

8. The positioning and stabilising structure as claimed in claim 7, wherein the first layer of material is a textile material.

9. The positioning and stabilising structure as claimed in claim 7, wherein the strap(s) have a multi-layer construction comprising the first layer of material and at least one additional layer of material.

10. The positioning and stabilising structure as claimed in claim 9, wherein the multi-layer construction comprises at least one additional layer and wherein the additional layer of material is a textile material.

11. The positioning and stabilising structure as claimed in claim 9, wherein the multi-layer construction comprises a layer of foam material.

12. The positioning and stabilising structure as claimed in claim 10, wherein the multi-layer construction comprises a plurality of layers that are laminated or glued together.

13. The positioning and stabilising structure as claimed in claim 1, wherein the strap has a patient contacting surface, and wherein the strap fastener component is located on the distal side of the patient contacting surface from the patient's face in use.

14. The positioning and stabilising structure as claimed in claim 4, further comprising a third strap and a fourth strap.

15. The positioning and stabilising structure as claimed in claim 14, wherein the first strap and the second strap provide a pair of lower straps for the positioning and stabilising structure and the third strap and the fourth strap provide a pair of upper straps for the positioning and stabilising structure.

16. The positioning and stabilising structure as claimed in claim 14, wherein the third strap and the fourth strap each comprises a connector configured to in use releasably attach the positioning and stabilising structure to a patient interface.

17. The positioning and stabilising structure as claimed in claim 16, wherein the connectors on the third strap and the fourth strap each comprises a magnetic fastener component configured to in use engage with a corresponding magnetic fastener component on a patient interface.

18. A treatment system, comprising
a patient interface to deliver a supply of pressurised breathable gas to one or more of a patient's airways, and
a positioning and stabilising structure;
wherein the positioning and stabilising structure comprises at least one strap having a strap fastener half, wherein the strap fastener half comprises a magnetic fastener component which is formed to the at least one strap, and
the patient interface comprises a patient interface fastener half which includes a magnetic fastener component, and further wherein
in use the magnetic fastener components together releasably attach the at least one strap to the patient interface.

19. A method of manufacturing a positioning and stabilising structure for a patient interface system, comprising the following steps in any order:
selecting a first layer of material;
positioning a magnetic fastener component and the first layer of material relative to each other;
attaching the magnetic fastener component to the first layer of material.

20. The method as claimed in claim 19, wherein the step of attaching the magnetic fastener component to first layer of material involves forming at least a portion of a strap for the positioning and stabilising structure.

21. The method as claimed in claim 20, wherein forming the strap involves laminating at least two layers of material to each other to form a multi-layer structure.

22. The method as claimed in claim 21, wherein laminating the at least two layers of material to each other forms the magnetic fastener component to the strap.

23. The method as claimed in claim 19, including the step of shaping the strap to have a desired shape.

24. The method as claimed in claim 23, wherein the step of shaping the strap includes cutting.

25. The method as claimed in claim 19, including the step of attaching the strap to another component of the positioning and stabilising structure.

26. The method as claimed in claim 25, wherein the step of attaching the strap to another component involves sewing to create a joint.
